(19) United States
(12) Patent Application Publication
(10) Pub. No.: US 2024/0066215 A1
(43) Pub. Date: Feb. 29, 2024

(54) DRUG DELIVERY DEVICE AND SYSTEM

(71) Applicant: AMGEN INC., Thousand Oaks, CA (US)

(72) Inventors: Scott R. Gibson, Thousand Oaks, CA (US); Mehran Mojarrad, Thousand Oaks, CA (US); Paul Daniel Faucher, Thousand Oaks, CA (US); Steven Edward Gorski, Poway, CA (US); Nicholas D.M. Prsha, Encinitas, CA (US); Eduardo Ho, Calsbad, CA (US); Rafi Muhammad Sufi, Simi Valley, CA (US)

(21) Appl. No.: 17/767,471

(22) PCT Filed: Oct. 14, 2020
(86) PCT No.: PCT/US20/55461

§ 371 Date: Apr. 8, 2022

Related U.S. Application Data
(60) Provisional application No. 62/923,224, filed on Oct. 18, 2019.
(60) Provisional application No. 62/923,876, filed on Oct. 21, 2019.
(60) Provisional application No. 62/923,904, filed on Oct. 21, 2019.
(60) Provisional application No. 62/925,576, filed on Oct. 24, 2019.

(51) Int. Cl. A61M 5/145; A61M 5/172

(52) U.S. Cl. CPC A61M 5/145; A61M 5/1723

(57) ABSTRACT

A drug delivery device for delivering a medicament includes a pump first housing, a pump second housing, an inlet fluid path, and an outlet fluid path. The pump first housing is at least partially supporting and/or surrounding a fluid displacement assembly. The pump second housing is at least partially supporting and/or surrounding a drive component for driving the fluid displacement assembly. The inlet fluid path is configured to deliver medicament to the fluid displacement assembly. The outlet fluid path is configured to receive medicament from the fluid displacement assembly. The pump first housing and the pump second housing are removably coupled with each other.

### CROSS-REFERENCE TO RELATED APPLICATIONS

Priority is claimed to U.S. Provisional Patent Application No. 62/923,224, filed Oct. 18, 2019, and to U.S. Provisional Patent Application No. 62/923,876, filed Oct. 21, 2019, and to U.S. Provisional Patent Application No. 62/923,904, filed Oct. 21, 2019, and to U.S. Provisional Patent Application No. 62/925,576, filed Oct. 24, 2019, and the entire contents of the each of the foregoing are hereby incorporated herein by reference.

### FIELD OF THE DISCLOSURE

The present disclosure generally relates to drug delivery devices, systems, and methods of use and, more particularly, to a pump and a system for long-term, continuous or semi-continuous, intravenous drug delivery.

### BACKGROUND

Drugs are administered to treat a variety of conditions and diseases. Intravenous (“IV”) therapy is a drug dosing process that delivers drugs directly into a patient's vein using an infusion contained in a delivery container such as an IV bag and tubing connected to a needle subsystem that fluidically communicates with the reservoir through the pump assembly collectively called infusion set. These drug dosings may be performed in a healthcare facility, or in some instances, at remote locations such as a patient's home. In certain applications, a drug delivery process may last for an extended period of time (e.g., for one hour or longer) or may include continuous or semi-continuous delivery of a drug over an extended period of time (e.g., for several hours, days, weeks, or longer). For many of these relatively long-term delivery requirements, a pump is often utilized to control and/or administer the drug to the patient. The pump may be coupled (physically, fluidly, and/or otherwise) to various components, such as a drug delivery container, supply lines, connection ports, and/or the patient.

It may be desirable to utilize a pump and/or overall system that is portable and/or wearable. It may also be desirable to utilize a pump and an overall system that minimizes patient inconvenience, minimizes the size and profile of the device and the overall system, minimizes the complexity of the device and overall system, minimizes the noise and vibration of the device, accommodates easy connection/disconnection and changeover of the infusion set, simplifies or automates priming of the line, accommodates easy delivery interruption and reestablishment based on required therapy and delivery profile, easily provides status of delivery and other important user information such as occlusion and volume of drug delivered or remaining in the reservoir, reduces the cost of the device and the overall system, increases the reliability and accuracy of the device and the overall system.

As described in more detail below, the present disclosure sets forth devices, systems, and methods for drug delivery embodying advantageous alternatives to existing devices, systems, and methods, and that may address one or more of the challenges or needs mentioned herein, as well as provide other benefits and advantages.

### BRIEF DESCRIPTION OF THE DRAWINGS

The above needs are at least partially met through provision of the systems and approaches for drug delivery device reconstitution described in the following detailed description, particularly when studied in conjunction with the drawings, wherein:

FIGS. 1a and 1b illustrate different views of an exemplary drug delivery device in accordance with various embodiments;

FIG. 2 illustrates a schematic internal view of an exemplary drug delivery device in accordance with various embodiments;

FIG. 3 illustrates an exploded view of an exemplary drug delivery device in accordance with various embodiments;

FIG. 4 illustrates an exploded view of an exemplary drive assembly for a drug delivery device in accordance with various embodiments;

FIG. 5 illustrates an exploded view of an exemplary pump head for a drug delivery device in accordance with various embodiments;

FIG. 6 illustrates an exploded view of an exemplary pressure sensor assembly and manifold assembly for a drug delivery device in accordance with various embodiments;

FIG. 7 illustrates an exploded view of an exemplary PCA and battery assembly for a drug delivery device in accordance with various embodiments;

FIG. 8 is a flow chart for an exemplary controller for a drug delivery device in accordance with various embodiments;

FIG. 9 illustrates an exemplary drug delivery system in accordance with various embodiments;

FIG. 10 is a flow chart for an exemplary method of using and/or priming a drug delivery system in accordance with various embodiments;

FIG. 11 illustrates an exemplary component of a drug delivery system in accordance with various embodiments; and

FIG. 12 illustrates two views of an exemplary drug delivery device in accordance with various embodiments;

FIG. 13 illustrates a view of an exemplary pump head for a drug delivery device in accordance with various embodiments;

FIG. 14 illustrates two views of an exemplary housing for a drug delivery device, in accordance with various embodiments;

FIG. 15 illustrates a view of an exemplary drug delivery device in a non-attached (left) and an attached configuration (right), in accordance with various embodiments;

FIG. 16 illustrates a view of an exemplary housing for a drug delivery device, in accordance with various embodiments;

FIG. 17 illustrates a view of an exemplary drug delivery device in a non-attached (left) and an attached configuration (right), in accordance with various embodiments;

FIG. 18 illustrates two views of an exemplary drug delivery device in a non-attached configuration, in accordance with various embodiments;

FIG. 19 illustrates two views of an exemplary drug delivery device in a non-attached configuration, in accordance with various embodiments;

FIG. 20 illustrates a view of an exemplary drug delivery device; and

FIGS. 21-23 each illustrates an exemplary drug delivery device pressure sensor and tubing in accordance with various embodiments.

Skilled artisans will appreciate that elements in the figures are illustrated for simplicity and clarity and have not necessarily been drawn to scale. For example, the dimensions and/or relative positioning of some of the elements in the figures may be exaggerated relative to other elements to help to improve understanding of various embodiments of the present invention. Also, common but well-understood elements that are useful or necessary in a commercially feasible embodiment are often not depicted in order to facilitate a less obstructed view of these various embodiments. It will further be appreciated that certain actions and/or steps may be described or depicted in a particular order of occurrence while those skilled in the art will understand that such specificity with respect to sequence is not actually required. It will also be understood that the terms and expressions used herein have the ordinary technical meaning as is accorded to such terms and expressions by persons skilled in the technical field as set forth above except where different specific meanings have otherwise been set forth herein.

### GENERAL DESCRIPTION

In some aspects, the present disclosure relates to a drug delivery device and, more particularly, to a fluid displacement assembly (such as a pump head) and a system for long-term, continuous or semi-continuous, intravenous drug delivery. In other aspects, the present disclosure relates to a drug delivery device such as a pump for long-term, continuous or semi-continuous, intravenous drug delivery. Under some conditions, a drug delivery process may last for an extended period of time (e.g., for one hour or longer), may include continuous or semi-continuous delivery of a drug over an extended period of time (e.g., for several hours, days, weeks, or longer), and may include delivery via an intravenous connection to a patient. In some other aspects, the present disclosure utilizes various features for potentially reduced noise, vibration, durability, and overall reliability while maintaining a relatively compact sized system that may be desirable or appropriate for extended, continuous or semi-continuous, intravenous delivery. In other aspects, the present disclosure utilizes various features, devices, systems, and methods for reducing and/or purging gas (e.g., air) from a fluid path of a drug delivery system. In still other aspects, the present disclosure utilizes various features for preventing undesired/accidental/unsuitable disconnection between device components during use.

For example, the present disclosure includes a drug delivery device for delivering a medicament, having a pump first housing at least partially supporting and/or surrounding a fluid displacement assembly; a pump second housing at least partially supporting and/or surrounding a drive component for driving the fluid displacement assembly; an inlet fluid path configured to deliver medicament to the fluid displacement assembly; an outlet fluid path configured to receive medicament from the fluid displacement assembly. The pump first housing and the pump second housing are removably coupled with each other. The device may further include a lock mechanism for selectively locking the pump first housing and the pump second housing in the coupled position. The lock may be electro-mechanical, electro-magnetic, mechanical, electronic, or any other suitable mechanism(s).

The pump first housing and the pump second housing may be removably coupled with each other along a first axis by a first component and along a second axis by a second component, wherein the first axis and the second axis are generally perpendicular to each other. The first component may be at least one magnet and the second component may be at least one slot-tab arrangement.

The drug delivery device may be utilized in a drug delivery system, including a medicament container containing a medicament; an inlet fluid path configured to receive the medicament from the medicament container; and an outlet fluid path configured to deliver the medicament to a patient. The drug delivery system may also include an adaptor for fluidly connecting at least two sections of the inlet fluid path with each other.

The drug delivery device may include at least one dampening component adjacent to an interface between the pump first housing and the pump second housing. The drug delivery device may have at least one sensor positioned along the inlet fluid path and configured to measure an operational parameter; and a controller workingly coupled with the at least one sensor and the drive component (either directly or indirectly, such as through interface board(s) for processing). The controller may be configured to adjust at least one parameter of the drive component based on input information received from the at least one sensor. The controller may include an encoder-fed, closed loop system.

The present disclosure discloses components, devices, and systems that may be able to have a secure, repeatable, and simple way to connect the durable portion of the pump (e.g., the durable pump controller or the durable housing) with the disposable portion of the pump (e.g., the pump head or the disposable housing), and optionally, to maintain the components in the connected configuration when desired. This not only makes the pump easier to use, especially for at-home use, but it also ensuring proper operation of the IV pump. At time of use, the user may install the pump head assembly onto the durable pump controller. As the two assemblies are connected, the eccentric hub slips into place, providing tactile feedback of positive engagement. Magnets may also provide additional tactile feedback when they latch the pump head assembly to the durable pump controller. The size and number of magnets are selected to ensure proper retention of the pump head assembly. The durable pump controller and pump head may also include another connection, such as a sliding connection. More specifically, the respective components may include a sliding dovetail connection. Because the tubing inlet and outlet of the infusion set are orthogonal to the dovetail connection, any forces applied to the infusion set from the pump are less likely to dislodge the pump head assembly.

The present disclosure discloses components, devices, and systems that may be able to mitigate noise and/or vibration from the drive motor. For example, the noise from the IV pump can be mitigated by isolating the motor from the rest of the controller case. Through over-molding, an elastomer can be integrated into the case body to isolate the vibration caused by the motor and reduce audible noise from the system. Additionally, or alternatively, by tuning the geometry of the over-molded elastomer based upon motor speeds, sprung weight, and durometer of the elastomer, this design can dampen the vibration from the motor and thereby reduce the sound experienced by the user.

Additionally, the present disclosure includes a drug delivery system for delivering a medicament, having a medicament container containing a medicament; a fluid path configured to receive the medicament from the medicament container; and a drug delivery device positioned along and/or adjacent to the fluid path; and an air vent fluidly coupled with the fluid path and configured to permit gas to exit the fluid path while resisting liquid from exiting the fluid path.

The drug delivery system may also include a drug delivery component and a connector for fluidly coupling the drug delivery device with the drug delivery component. The air vent and the connector may be separate components or they may be formed in a single component. The air vent may include a membrane that is at least substantially permeable by gas but that is at least substantially not permeable by liquid.

The present disclosure discloses components, devices, and systems that may be able to have a secure, repeatable, and simple way to prepare a system for drug delivery and/or use, including but not limited to priming a drug delivery system. This not only makes the pump easier and potentially safer and more effective to use, especially for at-home use, but it also helps ensure proper operation of the IV pump. The system may include gravity priming and/or priming by manually urging a liquid medicament into the flowpath. Neither type of priming requires the use of battery or motor power, increasing the longevity of operation, both types of priming can be accomplished relatively simply. For example, the system may include a hydrophobic filter along the fluid path, that allows gas to vent during priming while avoiding loss of fluid.

In most peristaltic pumps, IV fluid must run through the pump itself, which uses battery, can waste fluid, and be messy. It also requires that the pump be sterilized. In the IV pump-priming system disclosed herein, however, fluid only runs through the tubing in the consumable (such as the IV tubing and pump head); therefore, only the consumable needs to be sterilized. In addition, the IV bag can be gravity-fed to prime the line without use of the durable pump controller, which avoids the use of power in the priming procedure.

The hydrophobic membrane, may be integrated at the proximal end of the IV infusion set, allows for gravity priming using standard procedures. This avoids loss of drug product during the priming process. Additionally, the user may not need to turn the pump on, but simply to engage the IV infusion set and utilize an air vent, such as a standard slide valve, to facilitate gravity priming. IV fluid travels to the filter, priming the line, and stays in place at the filter until the user connects it to an infusion line. Once the system is primed, the IV can be administered by one of multiple methods, such as:
Hydrophobic filter is part of an integrated connector/filter component;
Hydrophobic filter, which is part of a cap, is removed after priming (and then connected to another IV line or a drug delivery component);
Hydrophobic filter is part of a cap which is pierced during installation; or
Hydrophobic filter is pierced during installation.

In other examples of the disclosure, a drug delivery device may include: a pump disposable housing at least partially supporting and/or surrounding a fluid displacement assembly; a pump durable housing at least partially supporting and/or surrounding a drive assembly for driving the fluid displacement assembly; a fluid path configured to permit the medicament to flow along the fluid path; a pressure sensor at least partially supported by and/or surrounded by the pump durable housing, wherein the pressure sensor is configured to measure a fluid pressure in the fluid path. The pump disposable housing and the pump durable housing may be removably coupled with each other. In such a design, the drug delivery device may have a pressure sensor interface within the durable pump controller that connects with the pump head assembly in the IV infusion set without compromising the fluid path. This sensor interface may provide at least the following: pressure readings of both the input (between IV bag and pump head assembly) and output (between pump head assembly and patient) lines. Additionally, other sensors can be implemented in the pump head assembly or durable pump controller assembly to allow a variety of enhanced control algorithms and state detection and feedback (e.g., temperature and air bubble detection).

By incorporating pressure-sensing component(s) in the durable pump controller (e.g., controller, electronics, battery, user interface) instead of in the consumable IV infusion set (e.g., pump head assembly, fluid path tubing, connectors/cannula, slide valve, air filter), the device may realize the following benefits:
Optimized and limited materials in contact with drug product
Fewer restrictions due to sterilization method compatibility
Improved sterility integrity
Reduction of components
Reduction of failure points and modes
Reduction of cost of goods (COG) in the consumable IV infusion set
Removal of electrical connection points
Reduction of assembly skill or complexity in the consumable IV infusion set
Smaller effective volume requiring sterilization

Additionally, the device may allow a healthcare practitioner to continue using the same durable pump controller when replacement of the IV bag is required. This is accomplished by using a sealed, sterile IV Infusion Set with each IV bag.

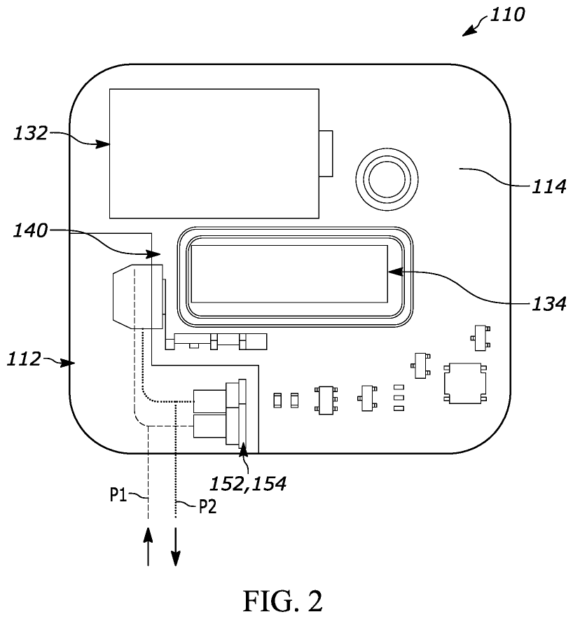

In another example, a drug delivery device may include first and second housings that are removably coupled with each other such that, when coupled with each other at least a portion of the drive assembly is received within at least a portion of the fluid displacement assembly. This design may incorporate a cartridge-like portion of the consumable that contains both the pump head as well as the fluid path interface (inlet and outlet) for the pressure sensor so that all connections are made at once, without user interaction (see FIG. 2).

In yet another example, a drug delivery device may have a storage configuration where first and second housings are not connected with each other and an operational configuration where they are connected with each other. This time-of-use connection increases the shelf life and integrity of the disposable set. The preservation of the continuous tube path may preserve the sterile integrity of the system. For example, in other peristaltic pumps, tubing that comes with the pump can become permanently compressed or kinked at the pinch location during extended storage. By delaying connection of the tube until time of use, deformation at the pinch point can be avoided, thus extending its shelf life. Additionally, the continuous tube path of the infusion set may not be interrupted by installation on the durable pump controller, which may be preferable to inline ambulatory IV pumps that require connection to the IV infusion set. The need to sterilize the durable pump controller may also be obviated.

In another example, a drug delivery device may include a pressure sensor positioned along and/or adjacent to the fluid path, wherein the pressure sensor is configured to measure a fluid pressure in the fluid path without being in fluid communication with the medicament. Such a pump may utilize pressure sensing in the IV pump platform without the need for fluid contact, thereby maintaining sterility in the durable pump controller. The design may permit a simpler and lower-cost IV infusion set that may be easier to manufacture. The design also reduces the materials that make contact with the drug product, while at the same time providing accurate delivery of drug product with real-time flow compensation and the built-in functionality of state detection (empty bag, occlusion detection, etc.)

As another aspect of the disclosure, an application of an infusion pump may be for use with a BiTE® therapy and may require the delivery of fluids intravenously to a patient over an extended period of time (˜4 weeks). Due to the prolonged therapy period, a patient may receive multiple IV bags, one at a time, to replace empty IV bags in order to continue therapy. In order to allow patients to change the IV bags themselves, the sequence of tasks required to change the IV bag and install a new one should be relatively easy to perform. For example, the method of connecting, maintaining the connection, and then releasing the connection between the durable pump control system and the removable pump head should be intuitive, user-friendly, and simple.

As a more specific example, the durable portion of the infusion pump may contain mechanical features to allow for correct alignment of the disposable pump head along the y and z direction.

As another example, the device may feature a software-controlled latching feature which, when the pump head is fully attached through mechanical means, will trigger a signal in the pump durable portion software to indicate successful attachment.

As yet another example, when the user would like to remove the disposable pump head, the user interface may allow for a release of the latching feature described above. For example, the user can press and hold a membrane switch/physical button on the pump durable system for a predetermined amount of time (e.g., 10 seconds). Once the pump head has been released from the latch, the user can then slide off the pump head without requiring a significant amount of force.

As another example, once the latching system is released, the pump would display a notification to the user that the pump head has been detached. In order to facilitate correct orientation for attachment, the durable portion of the pump and the disposable pump head may display markings indicating the fluid path.

### DETAILED DESCRIPTION OF THE FIGURES

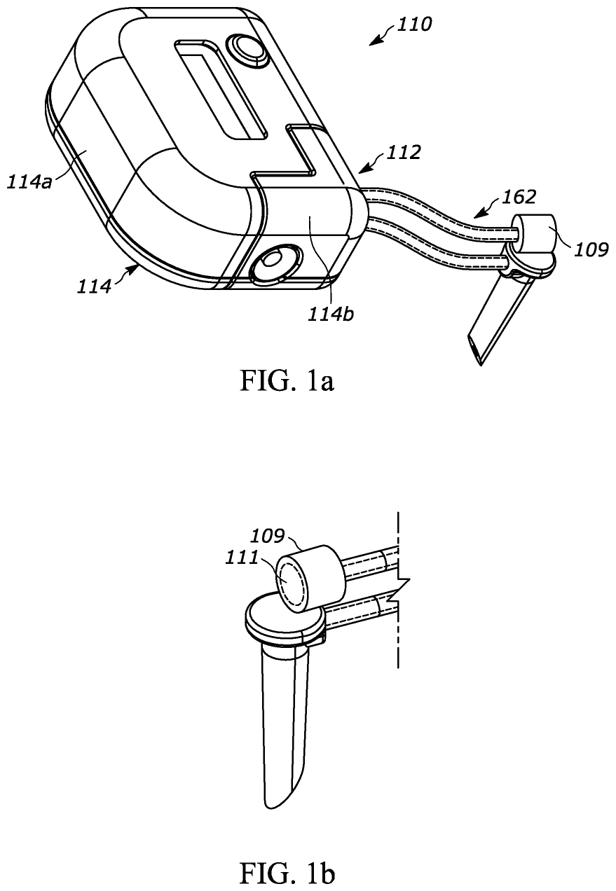

Turning to the figures, FIGS. 1a, 1b, and 2 shows a drug delivery device such as a pump 110 having, generally, a pump head 112 having a disposable housing 114b, a fluid flowpath 162, a durable housing 114a, a battery 132, a drive component or assembly such as a motor 140, a controller and display 134, and a pair of pressure sensors (e.g., inlet pressure transducer 152 and outlet pressure transducer 154), and air vent 109 (seen in FIGS. 1a and 1b) fluidly coupled with the flowpath 162 and configured to permit gas to exit the flowpath 162 while resisting liquid from exiting the flowpath 162. The two housing components 114a, 114b cooperate to define the overall housing 114. Additionally, the “durable housing 114a” is only preferably reusable/durable and may be disposable as suitable. Similarly, the “disposable housing 114b” may be reusable, although certain sterilization/refurbishment steps may be required or desirable.

The air vent 109 is preferably positioned near a proximal end of the flowpath 162 (e.g., the end of the flowpath 162 that engages the patient) so as to remove as much air as possible before delivering medicament to the patient. The air vent 109 may be a hydrophobic filter. For example, the air vent 109 may include a membrane 111 (seen in FIG. 1b) that is at least substantially permeable by gas but that is at least substantially not permeable by liquid.

As is further illustrated in FIG. 2, a medicament from a drug product container is able to travel along an inlet path P1 indicated with a dashed line, into the pump head 112, and out of the pump along an outlet path P2 indicated with a dotted line. In other words, the pump is able to urge the medicament through the pump head. The pump shown in FIG. 2 is a peristaltic pump but other suitable configurations may be used, such as a positive displacement pump. The pump head 112 shown in FIGS. 1 and 2 is a ring pump that utilizes a generally circular-shaped loop of tubing to create peristaltic forces. As a more specific example, the pump head has a component that pinches or otherwise occludes the ring-shaped tube section in a circular motion to urge fluid through the tube.

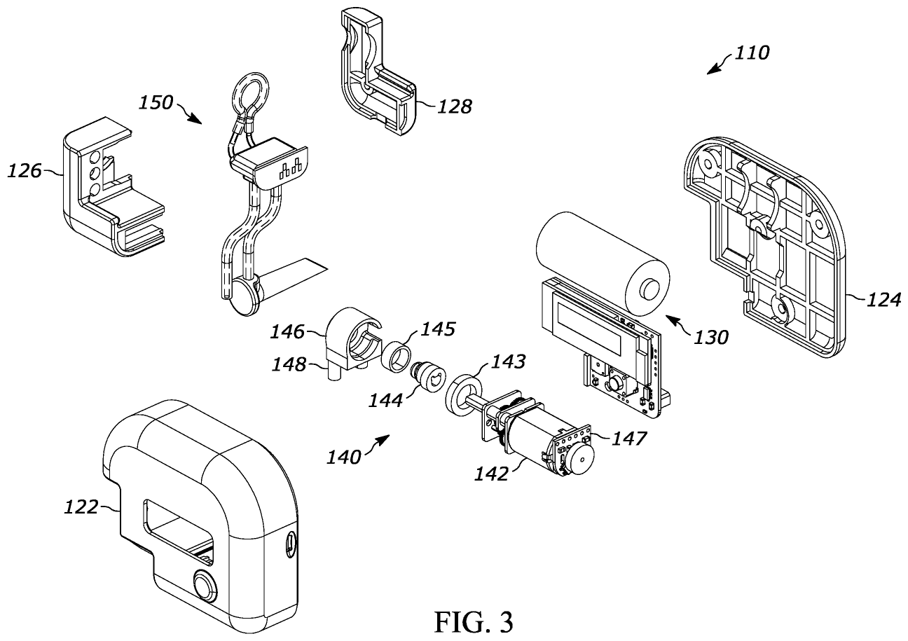

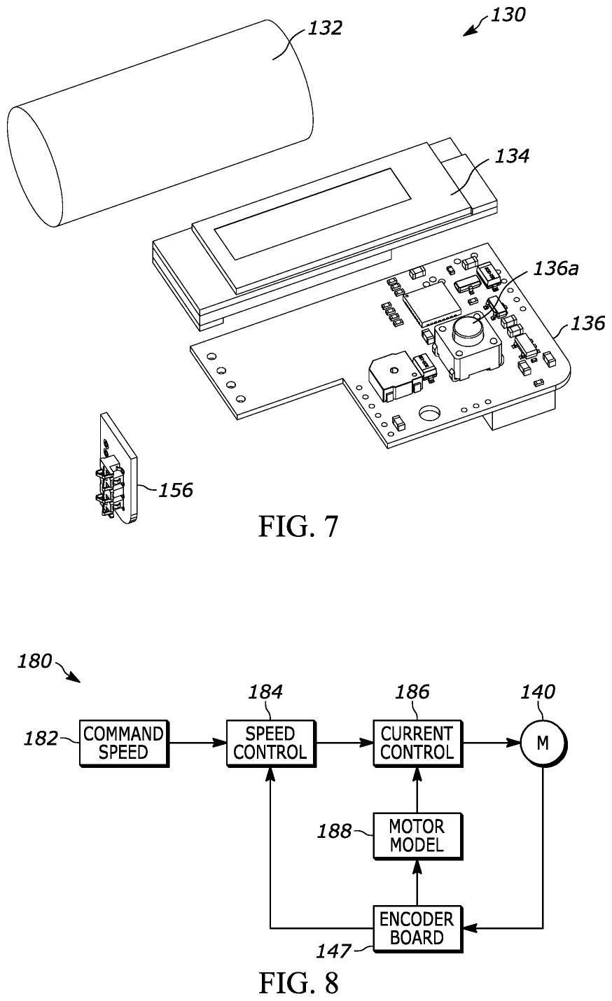

FIG. 3 shows an exploded view of the pump 110, including sub components of the housing 114, such as a controller front case 122, a controller rear case 124, a pump head front case 126, and a pump head rear case 128. These four components generally fit together to form at least the majority of the housing 114. These four components may be made of a generally rigid and lightweight material, such as plastic, a composite, or any other suitable material. The front/rear paired components (122, 124 on one hand, and 126, 128 on the other) may fit together via fasteners, snap-fit connections, an adhesive, or any other suitable coupling components/methods. A PCA and battery assembly 130 is at least partially contained within the housing 114, with a display screen 134 (FIG. 7) defining a portion of the housing 114.

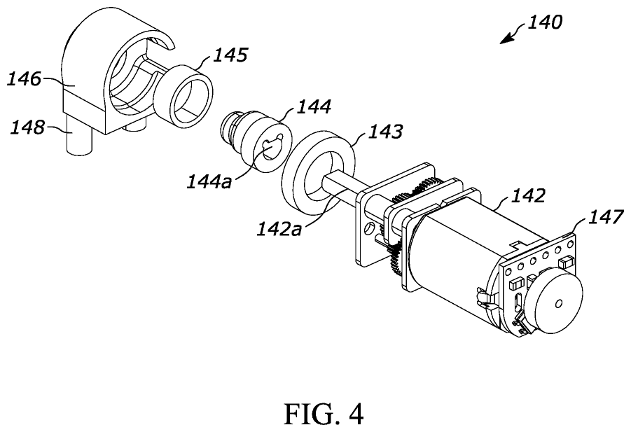

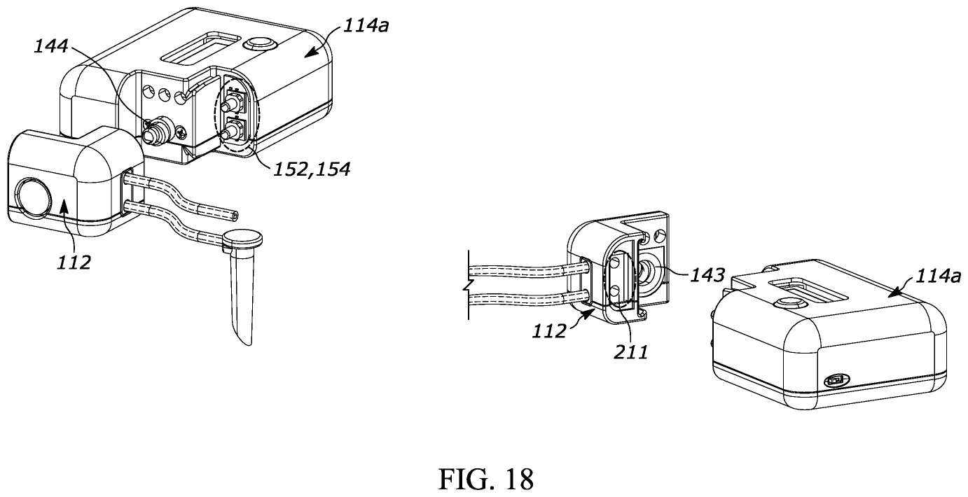

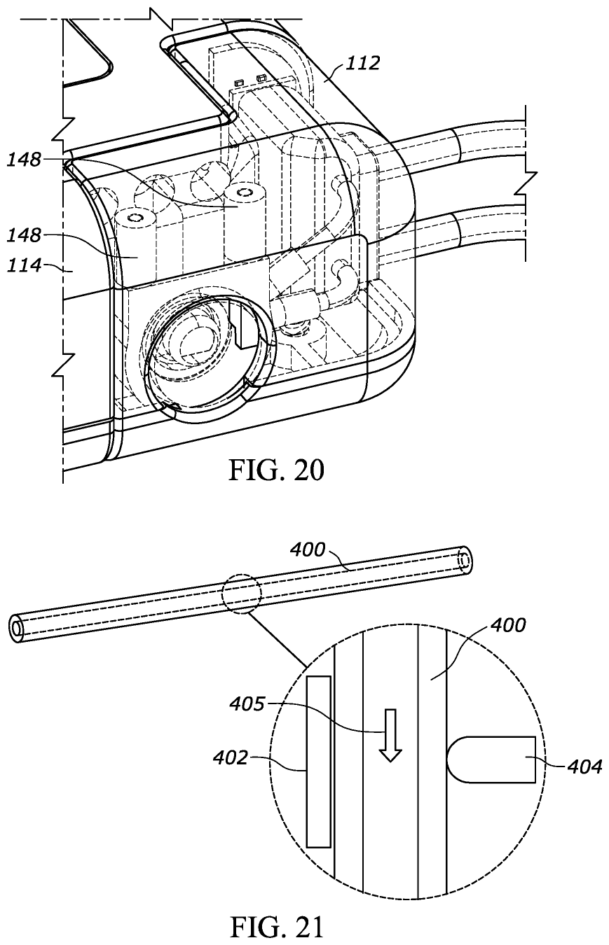

FIG. 3 further shows an exploded view of the drive assembly 140 (e.g., the motor assembly) and a tube set and pressure sensors 150. FIGS. 3 and 4 each show the exploded view of the drive assembly 140, which generally includes:
a motor 142 for providing rotational drive,
a retainer ring 143 (see also FIG. 18) for retaining other components in the housing (namely the tubes, as discussed more below) and/or for aligning the eccentric hub 144 (see also FIG. 18),
an eccentric hub 144 that utilizes a cam feature to generate peristalsis,
a sleeve bearing 145 that provides a barrier between the eccentric hub 144 and the tubing (such as the tube ring 158),
a pump race 146 for housing the circular-shaped tube section discussed above,
an encoder board 147 for measuring the actual speed of the motor for increased accuracy and precision, and
generally pliant/flexible isolation mounts 148 (seen also in FIG. 20) that prevent part misalignment, reduce drive torque/power, and provide compliance for head installation.

The isolation mounts 148 allow compliance to the pump head. As shown in FIG. 20, the removable pump head 112 may be compliantly mounted with respect to the durable portion 114 of the pump 110 via the isolation mounts 148, or other compliant mount components, to allow for easier installation and it to reduce motor voltage spikes due to component tolerancing. The isolation mounts 148 may be made of rubber or any other suitable material. As is shown in FIGS. 3 and 4, the eccentric hub 144 includes a key portion 144a that receives a correspondingly shaped drive shaft 142a. With reference to FIGS. 4 and 18-19, the eccentric hub 144, the drive shaft 142a, the motor 142, and the encoder board 147 are located within the durable portion 114a of the pump 112, whereas the retainer ring 143, the sleeve bearing 145, and the pump race 146 are all located within the removable pump head 112. When the pump head 112 is coupled with the durable portion of the pump 110, the eccentric hub 144 lines up with and is received within the retainer ring 143. During operation, as the drive shaft 142a of the motor rotates, the eccentric hub 144 has an eccentric feature that produces a cyclical outward force radially along the inner face of the sleeve bearing 145 as the hub 144 rotates, thereby applying an annular, outward force onto the circular-shaped tube section positioned within the pump race 146. The retainer ring 143 fits around the circumference of the eccentric hub 144. So configured, the retainer ring 143 retains the sleeve bearing 145 inside the removable pump head 112 and prevents it from falling out so that the removable pump head 112 can be attached to the durable portion of the pump 110 without losing the sleeve bearing 145. The sleeve bearing 145 rotates with the hub 144 and presses on the tube. The sleeve bearing 145 contacts the eccentric portion of the hub 144 and undulates with hub rotation while the retainer ring 143 is positioned in close proximity to, and optionally but not preferably in contact with, the circular portion of the hub 144 such that it does not spin or undulate. So as the eccentric hub 144 rotates, it may cause the sleeve bearing 145 to press on a relatively discrete portion of the circular-shaped tube section, thereby pinching and/or occluding that section of the tube. As mentioned, the retainer ring 143 may not move, but rather may provide a barrier to keep the tube from being pressed out of the pump race during use and to keep the tube in the pump race when not in use. As the eccentric hub 144 (and the sleeve bearing) rotates further, the portion of the outer surface of the sleeve bearing 145 that is pinching the tube “rolls” around the inside of the pump race 146 and urges fluid in the tube to travel away from the pump head 112.

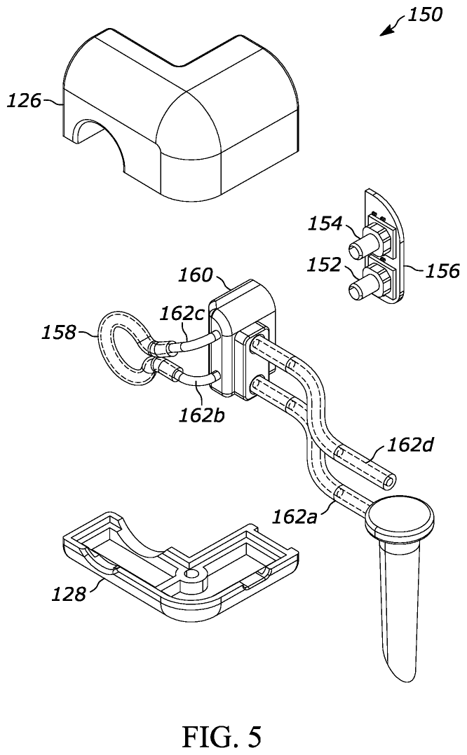

FIG. 5 shows the tube set and pressure sensors 150 in more detail, namely an exploded and enlarged view. FIG. 5 illustrates two sensors, namely inlet pressure transducer 152 and outlet pressure transducer 154, which measure fluid pressure in inlet and outlet portions of the flowpath 162a-d. The respective transducers 152, 154 shown in the figures make contact with the flow in the manifold 160 of the pump head 112. The tubing may be bonded to the manifold. As a more specific example, the transducers 152, 154 are electrically connected to the pump controller via sprung connector contacts and they directly measure the pressure in the flow at the inlet and outlet location. As an even more specific example, each transducer 152, 154 is electrically connected to a pressure transducer board 156 that is electrically connected to other electronic controls such as the motherboard (discussed below). For example, the transducers 152, 154 shown in the figures are each mounted on the pressure transducer board 156.

Each transducer 152, 154 shown in the figures may include a diaphragm, made from the same material as the tubing, placed inline on both the inlet and outlet tubes (162a, 162b). These diaphragms are located in the pump head 112 and make contact with a portion of the pump controller (e.g., the pressure transducer board) when the pump head assembly is installed via the pressure transducer board 156. At the point of diaphragm contact, load cells in the pump controller monitor variation in force exerted by the diaphragm which correlates to pressure changes in the flow. In this manner, the flow rate can be monitored at the inlet and outlet of the pump head 112 which provides the pressure sensor benefits discussed herein without introducing any new materials into drug contact.

Other or alternative types of pressure sensors may be utilized, such as non-contact pressure sensors design to provide the benefits of pressure sensors but without the risk of material non-compatibility.

In flow systems with a rigid fluid path, simply monitoring the speed of the pump head may be all that is necessary for precise flow control with a positive displacement pump: flow rate=[volume/revolution]*[revolutions/time]. However, in IV-based fluid systems, it may be beneficial to have the fluid path comprised of flexible tubing that expands and contracts with pressure, which subsequently affects the volume of product in peristaltic systems and may decrease effective accuracy. This pressure variation can occur simply from the variation in height of the IV bag with respect to the controller (pump), as well as from partial occlusion or other environmental influences. So, the effectiveness of flow control is dependent on assumptions of fluid input pressure.

Adding the ability for the system to monitor input and output pressure readings may allow the controller to adjust speed or duty cycle time of pump to compensate for variances in input pressure and maintain the desired flow rate of drug product. One possible embodiment of this design is a strain gage-based sensor that interfaces directly with the fluid path (tubing) wall. As the flexible material of the fluid path expands or contracts due to pressure variation, this relative movement is detected and interpreted as a pressure reading. The correlation of measured pressure to detected position is a function of geometry and material properties and can be optimized for the level of accuracy and resolution desired. This type of design is discussed below in more detail with respect to FIGS. 21-23.

Another embodiment utilizes a custom interface in the tubing set that comprises a rigid housing and flexible membrane with a variety of geometry (wall thickness, cross]sectional area, width, length, etc.) that can tailor the sensitivity of the interface area to internal fluid pressure. This variation can be accomplished by attaching this interface to standard tubing. This type of design is discussed below in more detail with respect to FIG. 20.

FIG. 5 also shows an example of the fluid flowpath 162a-d in more detail. For example, the fluid flowpath 162a-d may include an external tubing inlet side portion 162a, an internal tubing inlet side portion 162b, an internal tubing outlet side portion 162c, and an external tubing outlet side portion 162d. The various portions of tubing 162a-d may be integrally formed (i.e. a single piece of tubing), or they may be made of two or more sections of tubing that are fluidly connected with each other. The external tubing portions 162a, 162d shown in the figures are each formed of the same type and sized tubing as each other and potentially the same type and sized tubing as IV lines. The internal tubing portions 162b, 162c shown in the figures are each formed of a smaller diameter tube to facilitate pressure measurement. The flowpaths 162 also include a fluid displacement assembly, such as a ring tubing 158, i.e., the generally circular portion of tubing discussed above that is housed within the pump race 146. In one embodiment, the ring tubing 158 defines the boundary between the inlet fluid flowpath and the outlet fluid flowpath. As discussed above, the pump head 112 components shown in FIG. 5 are supported by the pump head front and rear case 126, 128 and the pump head 112 is removably coupled with the remainder of the pump structure. The pump head 112 may be disposable and the remainder pump structure may be reusable (e.g. “durable”).

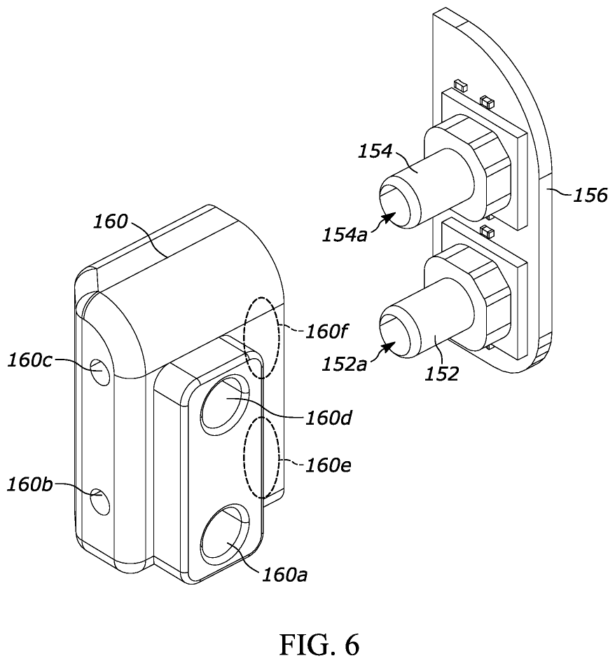

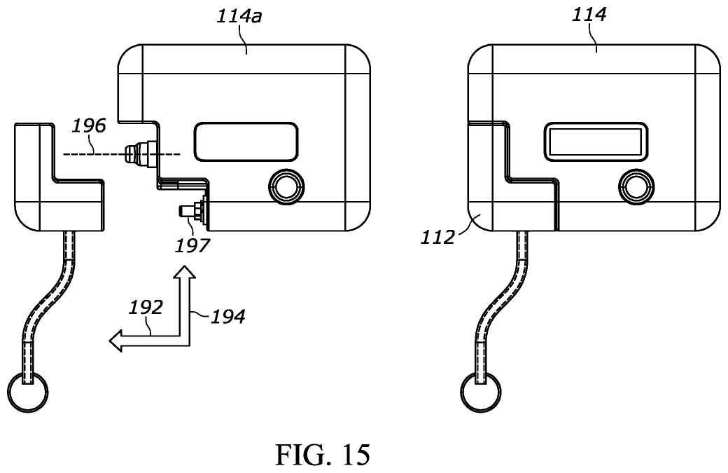

FIG. 6 shows an enlarged view of the tubing manifold 160 and the pressure sensors (152, 154, 156). The transducers 152, 154 are inserted within transducer ports (shown with dotted lines 160e and 160f) on the side of the manifold 160 so as to measure fluid pressure within the tubes that extend through the manifold 160. For example, the manifold includes manifold port external inlet 160a for receiving the external tubing inlet side portion 162a, manifold port internal inlet 160b for receiving the internal tubing inlet side portion 162b, manifold port internal outlet 160c for receiving the internal tubing outlet side portion 162c, and manifold port external outlet 160d for receiving the external tubing outlet side portion 162d. The transducer ports 160e, 160f are in-line with the other ports and are sized to receive the transducers 152, 154 and may be in fluid communication with the other ports. For example, ports 160a, 160b, and 160e are in fluid communication with each other such that the inlet fluid flow travels through tubing 162a, into contact with the diaphragm (the location of which is indicated by arrow 152a) of transducer 152, into tubing 162b, through the ring tubing 158, through tubing 162c, into contact with the diaphragm (the location of which is indicated by arrow 154a) of transducer 154, and into tubing 162d. While the diaphragms are disclosed as being located in the sensors 152, 154, in alternative versions, these diaphragms can be configured as integral components of the manifold 160. This may be of particular utility in connection with further embodiments where the sensors 152, 154 are disposed on the durable housing 114, see, e.g., FIGS. 15, 17, 18.

FIG. 7 shows the PCA and battery assembly 130, including a battery 132, an OLED display 134, a motherboard 136, and the pressure transducer board 156. As discussed above, the pressure transducer board 156 is electrically coupled with the motherboard 136 so the respective components can exchange inputs and outputs. The OLED display 134 may display user and/or pump information. The motherboard 136 may include a pushbutton 136a for operating the device, such as triggering a start or stop cycle. The motherboard 136 may host different functions and/or controls such as motor control; pulse with modulation (PWM) control; Proportional, Integral, Derivative (PID) control to stabilize the pump, sound control, user input control, and encoder board/speed control.

FIG. 8 shows a flowchart of one exemplary operation of a controller 180 of the pump 110 that has improved accuracy and/or precision. The controller 180 may include the motherboard 136 (seen in FIG. 7) and/or other components. For example, the controller 180 shown in FIG. 8 includes an encoder-fed, closed loop system. As a more specific example, the controller 180 shown in FIG. 8 includes an encoder board 147 for determining measured drive speed and a motor model 188 for determining a calculated drive speed. In this example, the controller is configured to adjust at least one parameter of the drive component based on the measured drive speed and the calculated drive speed. For example, during operation, the motherboard and/or a user input may dictate a desired speed for the motor 140, i.e., a “command speed” 182. The command speed 182 is then inputted into a “speed control” 184 component that may be coupled to or integrally formed in the motherboard 136. The speed control then sends input to the “current control” 186 which in turn sends a certain amount of current to the motor 140. An encoder board 147 is mounted to a portion of the motor 140 or adjacent to a portion of the motor 140 to measure the speed of a rotary portion of the motor. This measured speed information (e.g. measured drive speed) is then inputted back to the speed control 184. At the same time, the encoder board inputs the measured speed information to a motor model 188, which calculates a calculated (or predicted) drive speed based on operating conditions. The speed control 184 and the current control 186 each are able to receive and process these inputs and potentially vary their operation based on the same. For example, if the calculated speed from the encoder board 147 differs from the command speed 182, then the respective components may be able to adjust the current level sent to the motor 140 to more accurately and precisely operate at (or near) the command speed. The controller 180 may also receive inputs from the pressure sensors 152, 154 as part of the feedback/control system.

This feedback/control system may allow the pump 110 to operate at a high accuracy. For example the controller 180 may be configured such that the pump is able to deliver medicament at an accuracy rate of at least 95%. More specifically, the controller 180 may be configured such that the pump is able to deliver medicament at an accuracy rate of at least 97%. Even more specifically, the controller 180 may be configured such that the pump is able to deliver medicament at an accuracy rate of at least 98%. Even more specifically, the controller 180 may be configured such that the pump is able to deliver medicament at an accuracy rate of at least 99%. The controller 180 may be configured such that the pump is able to deliver medicament at one or more of these accuracy levels during delivery of a dose of the medicament having a volume of at least 200 milliliters or 250 milliliters. This feedback/control system may allow the pump 110 to operate at a high efficiency, thereby maximizing battery life, reducing device noise and vibration, reducing generated motor heat, and/or improving overall performance. The feedback/control system may allow the pump to operate at the accuracy levels discussed herein despite varying operating conditions, such as vertical height differential (positive or negative) between the pump and the drug product container. For example, the pump has been tested to maintain accuracy at +/−36 inches between the pump and the drug product container.

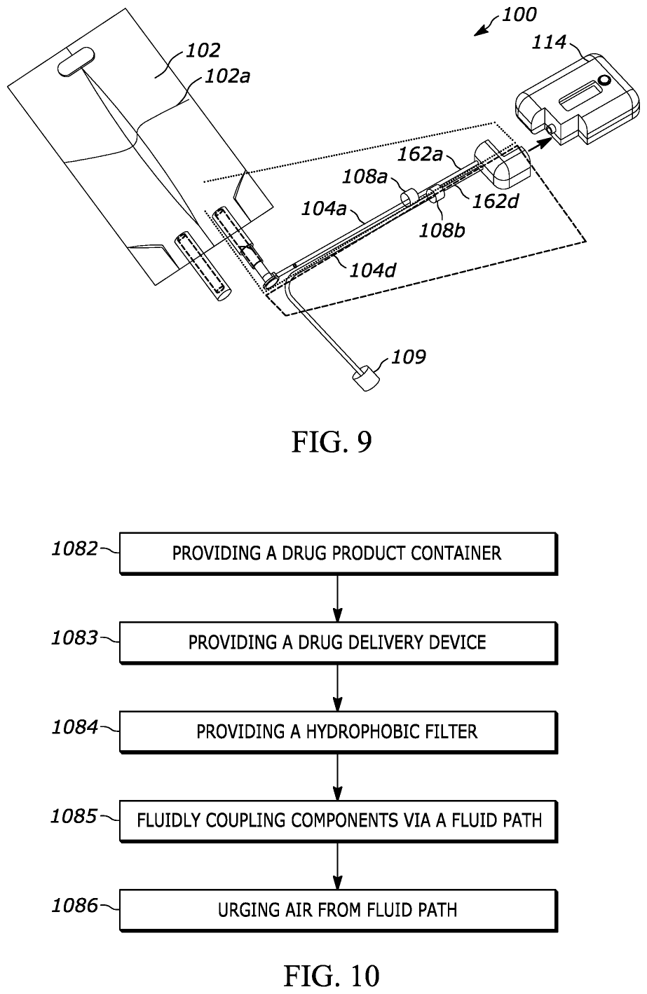

FIG. 9 shows an exemplary drug delivery assembly 100 (or “system”) for use with the pump 110. For example, the assembly 100 shown in FIG. 9 includes a drug product container 102 for containing a drug product 102a (or medicament), an IV input line 104a, an IV output line 104d, an optional pair of adaptors 108a, 108b, the air vent 109, and the tubing portions 162a, 162d leading to and from the pump 110. As a more specific example, the connection points may include quick-connect sterile connectors with respective sub-components that selectively mate with each other while maintaining sterility or another desirable cleanliness standard. For example, the quick-connect sterile connectors may snap or twist or screw together; they may have sheathed or covered components that become unsheathed or uncovered upon connection; and/or they may have Luer Lock or modified Luer Lock configurations. As another example, the connectors may include one or more stake connectors for coupling one of the tube 162 portions with an IV bag. The distal end of the IV output line 104d may also include or be coupled with a drug delivery connector (not shown) such as a needle, a Luer Lock component, or another suitable component. As shown in FIG. 9, an IV spikes may pierce the port of the drug container 102 to physically connect the drug product container to the fluid path assembly 160. The adaptors may be sterile quick-connect components. Example CSTD devices may include the OnGuard CSTD provided by B. Braun Medical Inc, BD PhaSeal CSTD components, Equashield CSTD, Codon CSTD, and the like. Further, non-closed system transfer devices may be used such as West Pharmaceuticals vial and bag adapters. Other examples are possible. The prefilled delivery container may include any number of delivery container adapters having different specifications (e.g., port sizes) to accommodate the use of different drug product vials.

FIG. 10 shows a flowchart of one exemplary operation for priming and/or preparing the drug delivery system 100 for use. For example, in step 1082 a user, health care provider, or other entity may provide a drug product container such as an IV bag; in step 1083 a user, health care provider, or other entity may provide a drug delivery device such as a pump; in step 1084 a user, health care provider, or other entity may provide a hydrophobic filter; in in step 1085 a user, health care provider, or other entity may fluidly connect the drug product container, the drug delivery device, and the hydrophobic filter; and in step 1086 a user, health care provider, or other entity may urge at least substantially all of the air from the fluid path. The air may be purged from the fluid path by any suitable urging step, such as a gravity priming (e.g. permitting gravity to cause the fluid to flow through the fluid path and purge the air from the same), or by squeezing the IV bag to facilitate purging.

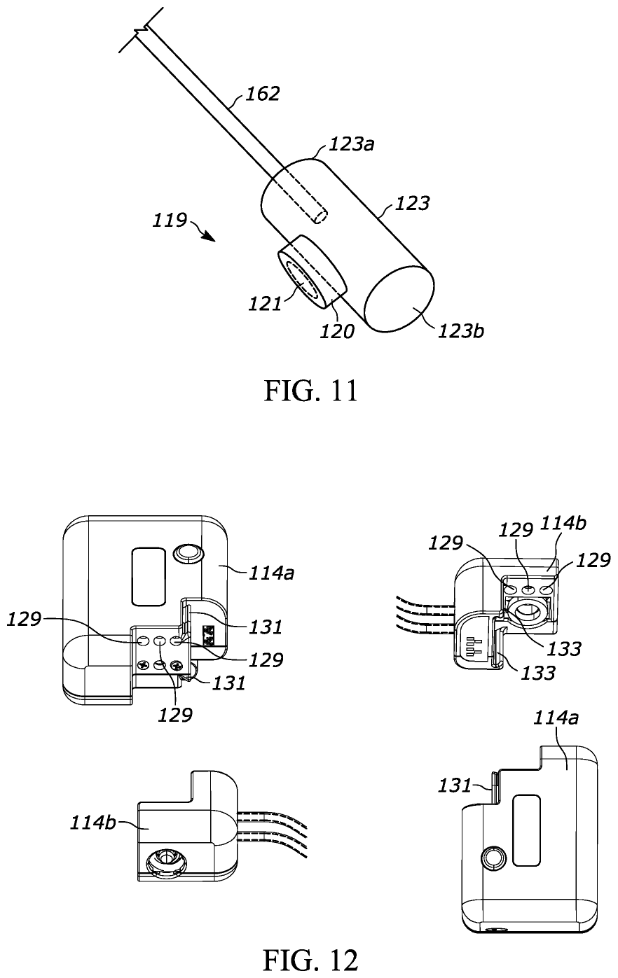

FIG. 11 shows an exemplary air vent 109 namely an integrated air vent/connector 119. The integrated component 119 includes an air vent 120 for facilitating purging of air from the fluid path and an adaptor 123 for fluidly connecting the IV tubing with another section of IV tube or a drug delivery component such as a needle. The air vent 120 includes a membrane 121 that is at least substantially permeable by gas but that is at least substantially not permeable by liquid. The adaptor has a first end 123a that is coupled with the IV tubing, a second end 123b that is configured to be coupled with another section of IV tube or a drug delivery component such as a needle.

Turning to FIG. 12, the durable housing 114a and disposable housing 114b are shown in a non-attached configuration, spaced apart from each other, and from different angled views. The two housing sections 114a, 114b are removably coupled with each other. For example, the two housing sections 114a, 114b shown in FIG. 12 are removably coupled with each other by a first component, for example at least one magnet 129, and a second component, for example at least one slot-tab arrangement 131/133. As a more specific example, each of the two housing sections 114a, 114b shown in FIG. 12 includes a plurality of magnets 129 that are generally aligned with each other to facilitate and promote selective coupling between the housing sections. Alternatively, it may be suitable to include one or magnets on only one of the two housing sections 114a, 114b, particularly if the at least one magnet is generally aligned with a ferrous-based metal component on the other of the two housing sections. As another example, any suitable component(s) may be used for coupling the two housing sections 114a, 114b, such as a press-fit arrangement, a hook connection, a locking arm, or any other suitable component(s).

As an additional feature or an alternative feature, the magnets 129 may serve as the lock mechanism for selectively locking the pump first housing and pump second housing in the coupled position. For example, the magnets 129 may be electromagnetically controlled such that they are selectively activated or de-activated to lock or unlock the housing components with each other.

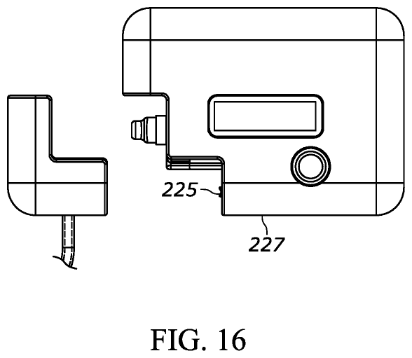

As another additional or an alternative feature, one of the housing components may include a latching mechanism 225 such as that depicted in FIG. 16. The latching mechanism 225 may serve as the lock mechanism for selectively locking the pump first housing and pump second housing in the coupled position. For example, the latching mechanism 225 may be electromechanically controlled such as a latching arm that moves from a locked and unlocked position based on an electronic signal from the controller. Alternatively, the latching mechanism 225 may be a mechanically-operated such as a pair of latching arms that move from a locked and unlocked position based on a release button 227 on the underside of the pump.

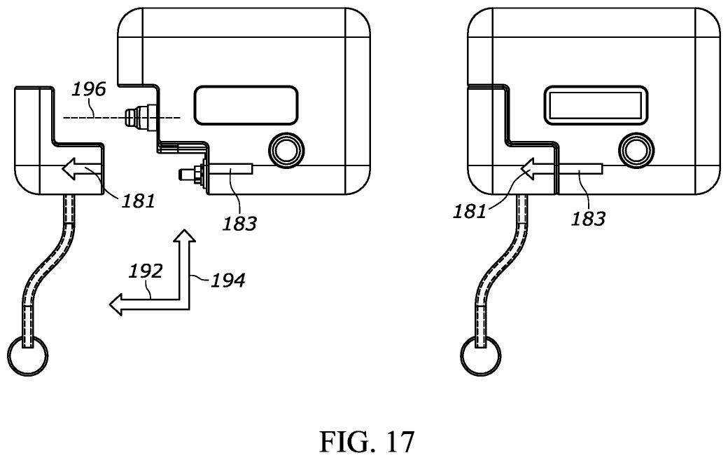

FIG. 17 also shows an alignment feature to assist the user in properly aligning the housing components. For example, marking 181 on the pump head and marking 183 on the durable housing line-up with each other when the housing components are properly aligned. Although a large red arrow is shown in FIG. 17, any suitable marking(s) may be used, including symbols or text. Alternatively or additionally, these markings may be on the bottom side (side of pump head which contains the fluid inlet and outlet tubing) or any other side of the pump head and durable housing.

FIG. 18 shows an exemplary design, with the pressure sensors 152, 154 located on the durable unit or reusable housing 114 rather than on the removable pump head 112.

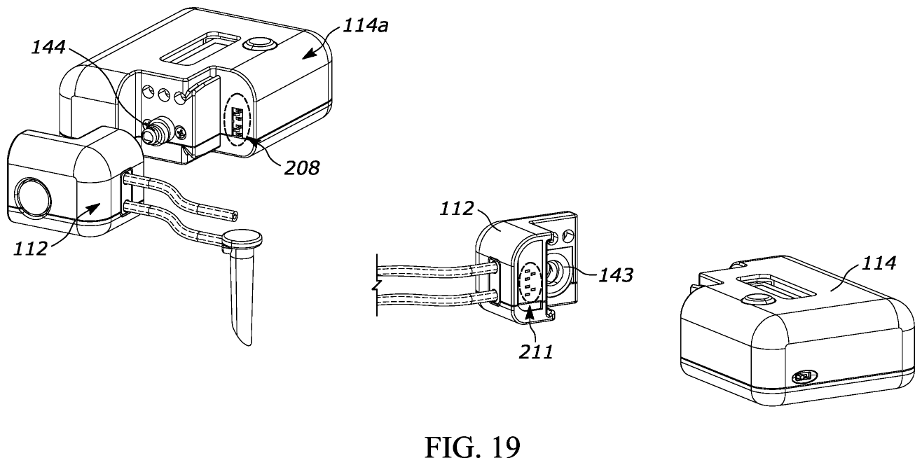

FIG. 19 shows an exemplary design with pressure sensors (not shown) located in the pump head, where the housing 114 includes sensor contacts 208 that cooperate with sensor board interface pads 211 located on the head 112. So configured, the pads 211 interact with controls in the housing 114 via the sensor contacts 208 to convey pressure information from the sensors.

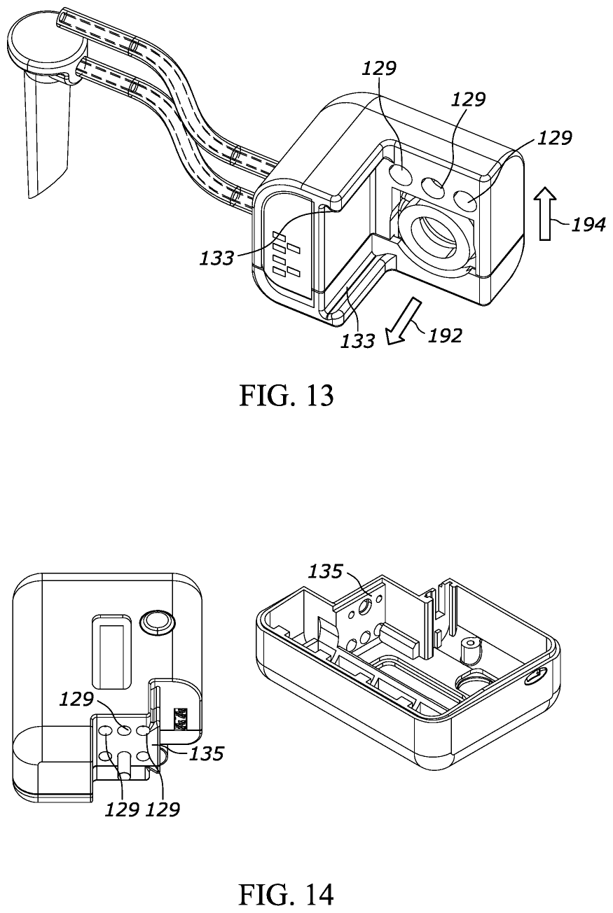

Turning back to FIG. 12, the two housing sections 114a, 114b shown in FIG. 12 include a second component for removably coupling the same. For example, the second component(s) may include a slot-tab arrangement 131/133. As a more specific example, the durable housing 114a shown in FIG. 12 includes a pair of elongated slots 131 that are configured to receive a pair of elongated tabs 133 on the disposable housing 114b. The slots 131 and tabs 133 may have a dovetail configuration, as shown in the figures, such as to prevent movement in any direction except for along axis 192 (FIG. 13). As a more specific example, the slots 131 are defined by a ridge running generally along axis 192 that is relatively smooth so as to permit sliding movement along the axis 192 but is also relatively strong to prevent or minimize deflection or relative movement between the two housing components.

As seen in FIGS. 15, the axis 192 is generally parallel with the drive axis 196 (FIG. 15). During operation, the drive axis 196 and the interaction between components directly and indirectly driven by the motor 142 will generally cause forces to act orthogonally to the drive axis 196. For example, during operation, the motor 142 causes the eccentric hub 144 to rotate and apply a force in a direction normal to the eccentric hub 144, i.e. outwardly towards the ring tubing 158. In other words, the forces generated from the motor 142 will act in directions perpendicular to the drive axis 196 and the axis 192. The design and orientation of the slot/tab 131/133 arrangement is therefore advantageous, as it generally secures the two housing components in all directions except along the axis 192, therefore improving the security of the interface between the housing components and minimizing relative movement therebetween. In other words, the orientation of the inlet and outlet tubes within the Pump Head Assembly is orthogonal to the strongest retention feature (e.g., the dovetail arrangement 131/133). This configuration may also reduce noise and vibration from component rattling or relative movement.

At the same time, the magnetic force, which is in the direction of the axis 192 is preferably strong enough to hold the components together during operation and when subject to gravitational forces, but is also preferably not too strong for a user to relatively easily overcome the magnetic forces when it is time to remove the pump head 112. The magnets 129 may also provide a tactile feel and/or an audible click as a signal/indicator to the user that the housing components are properly oriented and coupled.

The noise from the IV pump can be mitigated by isolating the motor from the rest of the controller case. For example, as shown in FIG. 14, at least one of the housing components 114a, 114b may include or be coupled with a dampening component 135 adjacent to the interface between the relative housing components. For example, in FIG. 14, the dampening component 135 is an overmolded component 135 forming a portion of the housing 114a. The component has openings for magnets and the drive shaft to extend through. The overmolded component 135 may be made of a material suitable for vibration and/or sound dampening, such as an elastomeric material. The overmolded component 135 may be integrated into the housing to isolate the vibration caused by the motor and reduce audible noise from the system. Alternatively, the component 135 may be a separate component that is coupled or connected to the housing. Additionally, or alternatively, the motor noise and/or vibration may be further limited by tuning the geometry of the over-molded elastomer based upon motor speeds, sprung weight, and durometer of the elastomer, this design can dampen the vibration from the motor and thereby reduce the sound experienced by the user.

The motor noise and/or vibration may be further limited through precise and accurate motor control, as discussed above with respect to FIG. 8. Additionally, or alternatively, the motor noise and/or vibration may be further limited by balancing the motor and transmission weight to the sprung weight on the end of the motor shaft, along with motor speed and output shaft speed. These may be key factors in tuning the system to minimize vibration. Additionally, or alternatively, the durometer and physical geometry of the housing component(s) are also key factors in tuning the system to minimize vibration.

The various components, devices, embodiments, and systems described may be advantageous over known components, devices, and systems for a number of reasons. For example, the pump designs and/or embodiments disclosed herein have a reduced, size, weight, and overall footprint compared to known pump designs. This advantage may offer dramatic quality of life and/or convenience for patients using the pump designs. As another example, the pump designs and/or embodiments disclosed herein may have an improved dose accuracy. As yet another example, the pump designs and/or embodiments disclosed herein may have a reduced complexity of the device and overall system. As yet another example, the pump designs and/or embodiments disclosed herein may have a reduced pump noise. As yet another example, the pump designs and/or embodiments disclosed herein may have a reduced cost of the device and the overall system. As yet another example, the pump designs and/or embodiments disclosed herein may have an increased reliability of the device and overall system. As yet another example, the pump designs and/or embodiments disclosed herein may have an increased product life of the device and overall system.

Another feature illustrated in FIG. 15 is that the pressure sensor 197 is at least partially supported by and/or surrounded by the pump durable housing 114a. As a more specific example, the pressure sensor 197 has a base portion supported by the durable housing 114a and another section extending away from the durable housing 114a for interaction and/or abutment with the fluid flow path when the housing sections 114a, 114b are coupled with each other.

Yet another feature illustrated in FIG. 15 is that the first housing and the pump second housing have a storage configuration (e.g., the figure on the left of FIG. 15) where they are not connected with each other and an operational configuration (e.g., the figure on the right of FIG. 15) where they are connected with each other. In one embodiment, when the pump first housing and the pump second housing are in the storage configuration, the pump first housing and the pump second housing are touching each other (but they are not connected). In other words, the respective dovetail components may be partially engaged while the magnets are not engaged and the housing components are spaced apart from each other along axis 192 so that the pump ring is not being compressed or pinched. The housing components are spaced apart from each other by a spacer (not shown) that is used for packaging purposes only. This arrangement may save space on shipping/packaging while maintaining the above-described advantages of a storage configuration.

FIG. 21 shows an alternative design for the type of pressure sensor utilized, namely one that can be utilized with a conventional IV tube 400 containing a flow 405 of medicament, for example, therethrough. The pressure sensor in FIG. 21 includes a tubing support 402 and a sensor contact 404 positioned in-line with the tubing support 402 such that as pressure increases or decreases and the IV tube 400 expands or contracts in small increments, the sensor contact 404 is able to measure the same and detect flow pressure. The sensor actuator and sensor electronics may be supported by and/or coupled with the durable housing 114 while the tubing section 400 and supported are supported by and/or coupled with the durable housing 114. However, alternative constructions may be utilized.

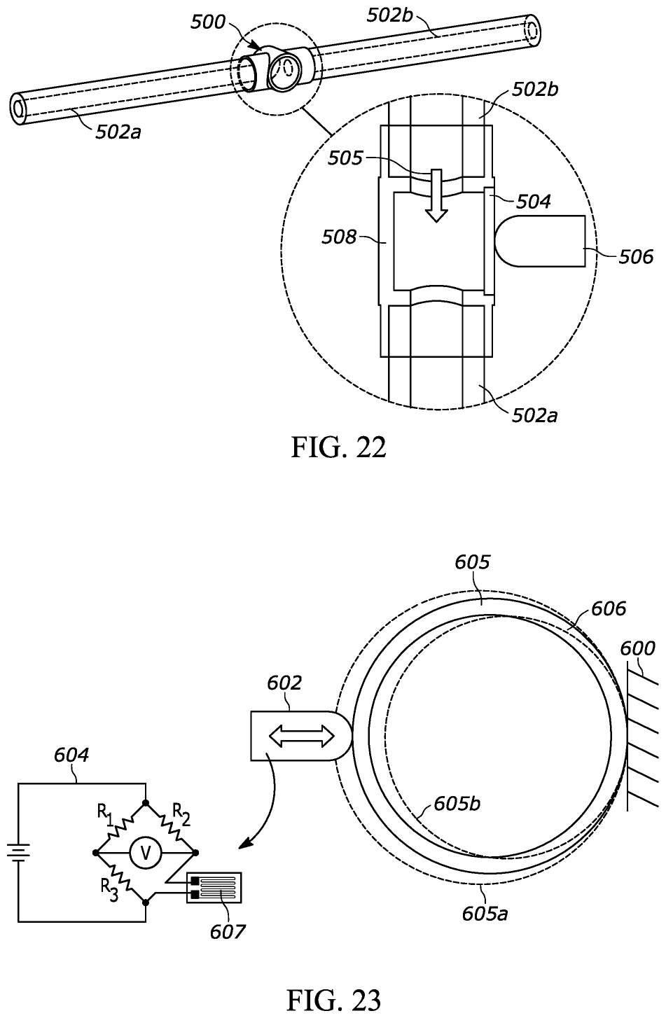

FIG. 22 shows another alternative design for the type of pressure sensor utilized, namely one that includes a connector 500 between two sections of conventional IV tubing 502a, 502b containing a flow 505 of medicament, for example, therethrough. The connector 500 has a rigid frame 508 that has a flowpath therethrough, a flexible diaphragm 504 fluidly connected with the flowpath such that the diaphragm 504 moves inward or outward based on the flow pressure therethrough, and a sensor contact 506 for measuring the position of the diaphragm 504.

FIG. 23 shows the schematic design for the type of non-contact pressure sensor utilized, namely one that a portion of the flowpath (IV tubing or diaphragm 605) 606 that is able to expand under increased pressure to an expanded cross-section 605a or contract under decreased pressure to a reduced cross-section 605b a support 600 as positional reference, a sensor actuator 602 to detect motion (expansion or contraction) of the tube 600 with respect to the reference support 600, and sensor electronics 604 including a strain gauge 607, for example, for responding to the sensor actuator 602, whereby the electronics communicate with the controller.

It may be desirable to utilize components that allow for fast/easy/sterile connections/disconnections. The fluid flowpath may be defined by a sterile single-use tubing system and valve system. The system may be used to provide intravenous, subcutaneous, intra-arterial, intramuscular, and/or epidural delivery approaches. By using the system, patient anxiety and or confusion may be reduced due to reduced preparation complexity and wait times caused by the drug preparation process.

In some examples, the system may be utilized with medicament in the form of a half-life extended bispecific T cell engager (BITE®). For example, the active pharmaceutical ingredient (“API”) may be between approximately 2 mcg and approximately 100 mcg, depending on the BiTE® and container size, which, may be in a powdered form (i.e., lyophilized) requiring reconstitution. In other examples, the drug product may be in liquid form and may not require reconstitution. Nonetheless, the system includes an accurate quantity of drug product, and thus does not require the need to add additional quantities thereto in a sterile environment. In some examples, the API may be in the form of a half-life extended (“HLE”) BiTE® and/or an IV-admin monoclonal antibody (“mAbs) as desired. These HLE BiTE®s include an antibody Fc region that advantageously provides different drug properties such as longer and extended half-lives. Accordingly, such APIs may be preferred due to their ability to maintain protective levels in the patient for relatively longer periods of time. Nonetheless, in other examples, the API may be in the form of a canonical-BITE® that is to be administered in a professional healthcare environment.

The medicament may also include other components such as an IVSS, saline solution, and/or a diluent. The IVSS may include polysorbate. In some examples, the IVSS formulation may include approximately 1.25 M lysine monohydrocholoride, 25 mM citric acid monohydrate, 0.1% (wN) polysorbate 80, and has a pH of approximately 7.0. In other examples, the IVSS 54 may include similar formulations, but also have a minimum of approximately 0.9% NaCl and approximately 0.001 to approximately 0.1% (wN) polysorbate 80. It is appreciated that different BiTE®s require different final percentages of IVSS 54 in the delivery container. This percentage may vary between approximately 0.5% to approximately 12% of the final volume in the delivery container. Further, citrate may increase the risk of glass delamination if filled in glass vials. In the event that citrate is necessary for drug product stabilization (determined on a per-product basis), the delivery containers may be constructed from CZ or other plastic compositions. Other examples of ingredients for suitable IVSSs are possible. Suitable IVSS concentrations protect against protein-plastic interactions and/or surface adsorption, and more specifically, in the lower end of the concentration range where even minor losses may potentially change the effective dose. The below table illustrates example component concentrations for varying IVSS concentrations:

TABLE 1
Component Concentrations with Varying IVSS Concentrations
(top column units are (V/v) % of IVSS

| IVSS COMPONENTS | 0.5 | 1.0 | 2.0 | 4.0 | 6.0 | 8.0 | 10.0 | 12.0 |
|---|---|---|---|---|---|---|---|---|
| Lysine monohydrochloride | 0.00625 | 0.0125 | 0.025 | 0.05 | 0.075 | 0.1 | 0.125 | 0.15 |
| (M) | | | | | | | | |
| Citrate Monohydrate (M) | 0.000125 | 0.00025 | 0.0005 | 0.001 | 0.0015 | 0.002 | 0.0025 | 0.003 |
| Polysorbate 80 (% w/v) | 0.0005 | 0.001 | 0.002 | 0.004 | 0.006 | 0.008 | 0.01 | 0.012 |

By providing the components in containers that are selectively connectable, it may be no longer necessary to prepare a needle and syringe assembly to inject one component into another container, to ensure that this prepared needle and syringe assembly is sterilized, and/or to ensure a correct volume or amounts of components are added together.

In some embodiments, the drug delivery system may have an integrated reconstitution subsystem onboard to dilute a lyophilized drug into a liquid form. In certain such embodiments, a diluent reservoir may be included for storing a diluent solution and a lyophilized reservoir may be included storing a lyophilized compound separate from the diluent solution. Furthermore, a fluid drive mechanism may be included for mixing the diluent solution in the diluent reservoir with the lyophilized compound in the lyophilized reservoir. In some embodiments, the fluid drive mechanism may transfer the diluent solution from the diluent reservoir into the lyophilized reservoir and/or provide any circulation and/or agitation needed to achieve full reconstitution. In some embodiments, an additional final reconstituted drug reservoir may be included and serve as a delivery reservoir from which the reconstituted drug is discharged into the patient; whereas, in other embodiments, the lyophilized reservoir may serve as the delivery reservoir. While the reconstitution subsystem may be physically integrated into the drug delivery system in certain embodiments, in other embodiments the reconstitution subsystem may constitute a separate unit which is in fluid communication with the drug delivery system. Having a separate unit may simplify the reconstitution process for healthcare providers in certain cases.

The drug product container may be in the form of an IV bag, a vial, a prefilled syringe, or similar container that includes a reconstitution container body defining an inner volume. The inner volume may be sterile. In some approaches, the reconstitution container adapter may also be a CSTD (or, in examples where the prefilled reconstitution container is in the form of a syringe, the container adapter may be a needle) that mates, engages, and/or couples to the vial adapter. Additionally or alternatively, the drug product can be bulk lyophilized and filled into a cartridge or container that is typically used to administer with an IV pump. If needed the dehydrated forms of IVSS, NaCl, and any other components needed for the final administered solution can be bulk lyo′ed and filled into the cassette for long term storage.

As previously noted, in some examples, the prefilled drug product container may be in the form of a prefilled syringe that contains the drug product. In these examples the drug product may be in the form of a liquid BiTE® formulation used in conjunction with a monoclonal antibody (mAb), In these examples, the drug product may be directly added to the delivery container without the use of a vial adapter system (such as the above-mentioned CSTDs) where more traditional needle-syringe injection/delivery into the container is preferred, which may advantageously simplify and/or improve supply chain and manufacturing control, and may further allow for more compact commercial packaging that takes up less space in storage systems at healthcare facilities. In these examples, the prefilled drug product vial may or may not need to be reconstituted prior to transferring the drug product to the delivery container.

The system may be distributed and/or sold as a common kit packaging, but other suitable distribution/packaging is suitable. The drug product may be in the form of a half-life extended bispecific T cell engager (BITE®), but other drug products are suitable. The diluent include water for injection (“WFI”), but other diluents may be suitable. The containers may be pliable bags, such as IV bags, but other containers may be suitable. In some examples, one or more of the containers is in the form of an IV drip bag constructed from a plastic or other material, e.g., 250 mL 0.9% Sodium Chloride IV bag constructed of a suitable material such as polyolefin, non-DEHP (diethylhexl phthalate), PVC, polyurethane, or EVA (ethylene vinyl acetate) and can be filled to a volume of approximately 270 mL to account for potential moisture loss over long-term storage.

In some examples, the prefilled delivery container is in the form of an IV drip bag constructed from a plastic or other material, e.g., 250 mL 0.9% Sodium Chloride IV bag constructed of a suitable material such as polyolefin, non-DEHP (diethylhexl phthalate), PVC, polyurethane, or EVA (ethylene vinyl acetate) and can be filled to a volume of approximately 270 mL to account for potential moisture loss over long-term storage. Other examples of suitable delivery containers are possible such as, for example, a glass bottle or container. Example suitable prefilled delivery containers are described in U.S. Appln. No. 62/804,447, filed on Feb. 12, 2019 and U.S. Appln. No. 62/877,286 filed on Jul. 22, 2019, the contents of each of which are incorporated by reference in their entirety.

The above description describes various devices, assemblies, components, subsystems and methods for use related to a drug delivery device. The devices, assemblies, components, subsystems, methods or drug delivery devices can further comprise or be used with a drug including but not limited to those drugs identified below as well as their generic and biosimilar counterparts. The term drug, as used herein, can be used interchangeably with other similar terms and can be used to refer to any type of medicament or therapeutic material including traditional and non-traditional pharmaceuticals, nutraceuticals, supplements, biologics, biologically active agents and compositions, large molecules, biosimilars, bioequivalents, therapeutic antibodies, polypeptides, proteins, small molecules and generics. Non-therapeutic injectable materials are also encompassed. The drug may be in liquid form, a lyophilized form, or in a reconstituted from lyophilized form. The following example list of drugs should not be considered as all-inclusive or limiting.

The drug will be contained in a reservoir. In some instances, the reservoir is a primary container that is either filled or pre-filled for treatment with the drug. The primary container can be a vial, a cartridge or a pre-filled syringe.

In some embodiments, the reservoir of the drug delivery device may be filled with or the device can be used with colony stimulating factors, such as granulocyte colony-stimulating factor (G-CSF). Such G-CSF agents include but are not limited to Neulasta® (pegfilgrastim, pegylated filgastrim, pegylated G-CSF, pegylated hu-Met-G-CSF) and Neupogen® (filgrastim, G-CSF, hu-MetG-CSF), UDENYCA® (pegfilgrastim-cbqv), Ziextenzo® (LA-EP2006; pegfilgrastim-bmez), or FULPHILA (pegfilgrastim-bmez).

In other embodiments, the drug delivery device may contain or be used with an erythropoiesis stimulating agent (ESA), which may be in liquid or lyophilized form. An ESA is any molecule that stimulates erythropoiesis. In some embodiments, an ESA is an erythropoiesis stimulating protein. As used herein, “erythropoiesis stimulating protein” means any protein that directly or indirectly causes activation of the erythropoietin receptor, for example, by binding to and causing dimerization of the receptor. Erythropoiesis stimulating proteins include erythropoietin and variants, analogs, or derivatives thereof that bind to and activate erythropoietin receptor; antibodies that bind to erythropoietin receptor and activate the receptor; or peptides that bind to and activate erythropoietin receptor. Erythropoiesis stimulating proteins include, but are not limited to, Epogen® (epoetin alfa), Aranesp® (darbepoetin alfa), Dynepo® (epoetin delta), Mircera® (methyoxy polyethylene glycol-epoetin beta), Hematide®, MRK-2578, INS-22, Retacrit® (epoetin zeta), Neorecormon® (epoetin beta), Silapo® (epoetin zeta), Binocrit® (epoetin alfa), epoetin alfa Hexal, Abseamed® (epoetin alfa), Ratioepo® (epoetin theta), Eporatio® (epoetin theta), Biopoin® (epoetin theta), epoetin alfa, epoetin beta, epoetin iota, epoetin omega, epoetin delta, epoetin zeta, epoetin theta, and epoetin delta, pegylated erythropoietin, carbamylated erythropoietin, as well as the molecules or variants or analogs thereof.

Among particular illustrative proteins are the specific proteins set forth below, including fusions, fragments, analogs, variants or derivatives thereof: OPGL specific antibodies, peptibodies, related proteins, and the like (also referred to as RAN KL specific antibodies, peptibodies and the like), including fully humanized and human OPGL specific antibodies, particularly fully humanized monoclonal antibodies; Myostatin binding proteins, peptibodies, related proteins, and the like, including myostatin specific peptibodies; IL-4 receptor specific antibodies, peptibodies, related proteins, and the like, particularly those that inhibit activities mediated by binding of IL-4 and/or IL-13 to the receptor; Interleukin 1-receptor 1 (“IL1-R1”) specific antibodies, peptibodies, related proteins, and the like; Ang2 specific antibodies, peptibodies, related proteins, and the like; NGF specific antibodies, peptibodies, related proteins, and the like; CD22 specific antibodies, peptibodies, related proteins, and the like, particularly human CD22 specific antibodies, such as but not limited to humanized and fully human antibodies, including but not limited to humanized and fully human monoclonal antibodies, particularly including but not limited to human CD22 specific IgG antibodies, such as, a dimer of a human-mouse monoclonal hLL2 gamma-chain disulfide linked to a human-mouse monoclonal hLL2 kappa-chain, for example, the human CD22 specific fully humanized antibody in Epratuzumab, CAS registry number 501423-23-0; IGF-1 receptor specific antibodies, peptibodies, and related proteins, and the like including but not limited to anti-IGF-1R antibodies; B-7 related protein 1 specific antibodies, peptibodies, related proteins and the like (“B7RP-1” and also referring to B7H2, ICOSL, B7h, and CD275), including but not limited to B7RP-specific fully human monoclonal IgG2 antibodies, including but not limited to fully human IgG2 monoclonal antibody that binds an epitope in the first immunoglobulin-like domain of B7RP-1, including but not limited to those that inhibit the interaction of B7RP-1 with its natural receptor, ICOS, on activated T cells; IL-15 specific antibodies, peptibodies, related proteins, and the like, such as, in particular, humanized monoclonal antibodies, including but not limited to HuMax IL-15 antibodies and related proteins, such as, for instance, 145c7; IFN gamma specific antibodies, peptibodies, related proteins and the like, including but not limited to human IFN gamma specific antibodies, and including but not limited to fully human anti-IFN gamma antibodies; TALL-1 specific antibodies, peptibodies, related proteins, and the like, and other TALL specific binding proteins; Parathyroid hormone (“PTH”) specific antibodies, peptibodies, related proteins, and the like; Thrombopoietin receptor (“TPO-R”) specific antibodies, peptibodies, related proteins, and the like;Hepatocyte growth factor (“HGF”) specific antibodies, peptibodies, related proteins, and the like, including those that target the HGF/SF:cMet axis (HGF/SF:c-Met), such as fully human monoclonal antibodies that neutralize hepatocyte growth factor/scatter (HGF/SF); TRAIL-R2 specific antibodies, peptibodies, related proteins and the like; Activin A specific antibodies, peptibodies, proteins, and the like; TGF-beta specific antibodies, peptibodies, related proteins, and the like; Amyloid-beta protein specific antibodies, peptibodies, related proteins, and the like; c-Kit specific antibodies, peptibodies, related proteins, and the like, including but not limited to proteins that bind c-Kit and/or other stem cell factor receptors; OX40L specific antibodies, peptibodies, related proteins, and the like, including but not limited to proteins that bind OX40L and/or other ligands of the OX40 receptor; Activase® (alteplase, tPA); Aranesp® (darbepoetin alfa) Erythropoietin [30-asparagine, 32-threonine, 87-valine, 88-asparagine, 90-threonine], Darbepoetin alfa, novel erythropoiesis stimulating protein (NESP); Epogen® (epoetin alfa, or erythropoietin); GLP-1, Avonex® (interferon beta-1a); Bexxar® (tositumomab, anti-CD22 monoclonal antibody); Betaseron® (interferon-beta); Campath® (alemtuzumab, anti-CD52 monoclonal antibody); Dynepo® (epoetin delta); Velcade® (bortezomib); MLN0002 (anti-α4β7 mAb); MLN1202 (anti-CCR2 chemokine receptor mAb); Enbrel® (etanercept, TNF-receptor/Fc fusion protein, TNF blocker); Eprex® (epoetin alfa); Erbitux® (cetuximab, anti-EGFR/HER1/c-ErbB-1); Genotropin® (somatropin, Human Growth Hormone); Herceptin® (trastuzumab, anti-HER2/neu (erbB2) receptor mAb); Kanjinti™ (trastuzumab-anns) anti-HER2 monoclonal antibody, biosimilar to Herceptin®, or another product containing trastuzumab for the treatment of breast or gastric cancers; Humatrope® (somatropin, Human Growth Hormone); Humira® (adalimumab); Vectibix® (panitumumab), Xgeva® (denosumab), Prolia® (denosumab), Immunoglobulin G2 Human Monoclonal Antibody to RANK Ligand, Enbrel® (etanercept, TNF-receptor/Fc fusion protein, TNF blocker), Nplate® (romiplostim), rilotumumab, ganitumab, conatumumab, brodalumab, insulin in solution; Infergen® (interferon alfacon-1); Natrecor® (nesiritide; recombinant human B-type natriuretic peptide (hBNP); Kineret® (anakinra); Leukine® (sargamostim, rhuGM-CSF); LymphoCide® (epratuzumab, anti-CD22 mAb); Benlysta™ (lymphostat B, belimumab, anti-BlyS mAb); Metalyse® (tenecteplase, t-PA analog); Mircera® (methoxy polyethylene glycol-epoetin beta); Mylotarg® (gemtuzumab ozogamicin); Raptiva® (efalizumab); Cimzia® (certolizumab pegol, CDP 870); Soliris™ (eculizumab); pexelizumab (anti-C5 complement); Numax® (MEDI-524); Lucentis® (ranibizumab); Panorex® (17-1A, edrecolomab); Trabio® (lerdelimumab); TheraCim hR3 (nimotuzumab); Omnitarg (pertuzumab, 2C4); Osidem® (IDM-1); OvaRex® (B43.13); Nuvion® (visilizumab); cantuzumab mertansine (huC242-DM1); NeoRecormon® (epoetin beta); Neumega® (oprelvekin, human interleukin-11); Orthoclone OKT3® (muromonab-CD3, anti-CD3 monoclonal antibody); Procrit® (epoetin alfa); Remicade® (infliximab, anti-TNFα monoclonal antibody); Reopro® (abciximab, anti-GP Iib/Iiia receptor monoclonal antibody); Actemra® (anti-IL6 Receptor mAb); Avastin® (bevacizumab), HuMax-CD4 (zanolimumab); Mvasi™ (bevacizumab-awwb); Rituxan® (rituximab, anti-CD20 mAb); Tarceva® (erlotinib); Roferon-A®-(interferon alfa-2a); Simulect® (basiliximab); Prexige® (lumiracoxib); Synagis® (palivizumab); 145c7-CHO (anti-IL15 antibody, see U.S. Pat. No. 7,153,507); Tysabri® (natalizumab, anti-α4integrin mAb); Valortim® (MDX-1303, anti-B. anthracis protective antigen mAb); ABthrax™ Xolair® (omalizumab); ETI211 (anti-MRSA mAb); IL-1 trap (the Fc portion of human IgG1 and the extracellular domains of both IL-1 receptor components (the Type I receptor and receptor accessory protein)); VEGF trap (Ig domains of VEGFR1 fused to IgG1 Fc); Zenapax® (daclizumab); Zenapax® (daclizumab, anti-IL-2Ra mAb); Zevalin® (ibritumomab tiuxetan); Zetia® (ezetimibe); Orencia® (atacicept, TACI-Ig); anti-CD80 monoclonal antibody (galiximab); anti-CD23 mAb (lumiliximab); BR2-Fc (huBR3/huFc fusion protein, soluble BAFF antagonist); CNTO 148 (golimumab, anti-TNFα mAb); HGS-ETR1 (mapatumumab; human anti-TRAIL Receptor-1 mAb); HuMax-CD20 (ocrelizumab, anti-CD20 human mAb); HuMax-EGFR (zalutumumab); M200 (volociximab, anti-α5β1 integrin mAb); MDX-010 (ipilimumab, anti-CTLA-4 mAb and VEGFR-1 (IMC-18F1); anti-BR3 mAb; anti-C. difficile Toxin A and Toxin B C mAbs MDX-066 (CDA-1) and MDX-1388); anti-CD22 dsFv-PE38 conjugates (CAT-3888 and CAT-8015); anti-CD25 mAb (HuMax-TAC); anti-CD3 mAb (NI-0401); adecatumumab; anti-CD30 mAb (MDX-060); MDX-1333 (anti-IFNAR); anti-CD38 mAb (HuMax CD38); anti-CD40L mAb; anti-Cripto mAb; anti-CTGF Idiopathic Pulmonary Fibrosis Phase I Fibrogen (FG-3019); anti-CTLA4 mAb; anti-eotaxin1 mAb (CAT-213); anti-FGF8 mAb; anti-ganglioside GD2 mAb; anti-ganglioside GM2 mAb; anti-GDF-8 human mAb (MYO-029); anti-GM-CSF Receptor mAb (CAM-3001); anti-HepC mAb (HuMax HepC); anti-IFNα mAb (MEDI-545, MDX-198); anti-IGF1R mAb; anti-IGF-1R mAb (HuMax-Inflam); anti-IL12 mAb (ABT-874); anti-IL12/1L23 mAb (CNTO 1275); anti-IL13 mAb (CAT-354); anti-IL2Ra mAb (HuMax-TAC); anti-IL5 Receptor mAb; anti-integrin receptors mAb (MDX-018, CNTO 95); anti-IP10 Ulcerative Colitis mAb (MDX-1100); BMS-66513; anti-Mannose Receptor/hCG8 mAb (MDX-1307); anti-mesothelin dsFv-PE38 conjugate (CAT-5001); anti-PD1mAb (MDX-1106 (ONO-4538)); anti-PDGFRa antibody (IMC-3G3); anti-TGFβ mAb (GC-1008); anti-TRAIL Receptor-2 human mAb (HGS-ETR2); anti-TWEAK mAb; anti-VEGFR/Flt-1 mAb; and anti-ZP3 mAb (HuMax-ZP3).

In some embodiments, the drug delivery device may contain or be used with a sclerostin antibody, such as but not limited to romosozumab, blosozumab, BPS 804 (Novartis), Evenity™ (romosozumab-aqqg), another product containing romosozumab for treatment of postmenopausal osteoporosis and/or fracture healing and in other embodiments, a monoclonal antibody (IgG) that binds human Proprotein Convertase Subtilisin/Kexin Type 9 (PCSK9). Such PCSK9 specific antibodies include, but are not limited to, Repatha® (evolocumab) and Praluent® (alirocumab). In other embodiments, the drug delivery device may contain or be used with rilotumumab, bixalomer, trebananib, ganitumab, conatumumab, motesanib diphosphate, brodalumab, vidupiprant or panitumumab. In some embodiments, the reservoir of the drug delivery device may be filled with or the device can be used with IMLYGIC® (talimogene laherparepvec) or another oncolytic HSV for the treatment of melanoma or other cancers including but are not limited to OncoVEXGALV/CD; OrienX010; G207, 1716; NV1020; NV12023; NV1034; and NV1042. In some embodiments, the drug delivery device may contain or be used with endogenous tissue inhibitors of metalloproteinases (TIMPs) such as but not limited to TIMP-3. In some embodiments, the drug delivery device may contain or be used with Aimovig® (erenumab-aooe), anti-human CGRP-R (calcitonin gene-related peptide type 1 receptor) or another product containing erenumab for the treatment of migraine headaches. Antagonistic antibodies for human calcitonin gene-related peptide (CGRP) receptor such as but not limited to erenumab and bispecific antibody molecules that target the CGRP receptor and other headache targets may also be delivered with a drug delivery device of the present disclosure. Additionally, bispecific T cell engager (BITE®) antibodies such as but not limited to BLINCYTO® (blinatumomab) can be used in or with the drug delivery device of the present disclosure. In some embodiments, the drug delivery device may contain or be used with an APJ large molecule agonist such as but not limited to apelin or analogues thereof. In some embodiments, a therapeutically effective amount of an anti-thymic stromal lymphopoietin (TSLP) or TSLP receptor antibody is used in or with the drug delivery device of the present disclosure. In some embodiments, the drug delivery device may contain or be used with Avsola™ (infliximab-axxq), anti-TNF α monoclonal antibody, biosimilar to Remicade® (infliximab) (Janssen Biotech, Inc.) or another product containing infliximab for the treatment of autoimmune diseases. In some embodiments, the drug delivery device may contain or be used with Kyprolis® (carfilzomib), (25)-N-((5)-1-((S)-4-methyl-1-((R)-2-methyloxiran-2-yl)-1-oxopentan-2-ylcarbamoyl)-2-phenylethyl)-2-((S)-2-(2-morpholinoacetamido)-4-phenylbutanamido)-4-methylpentanamide, or another product containing carfilzomib for the treatment of multiple myeloma. In some embodiments, the drug delivery device may contain or be used with Otezla® (apremilast), N-[2-[(15)-1-(3-ethoxy-4-methoxyphenyl)-2-(methylsulfonyl)ethyl]-2,3-dihydro-1,3-dioxo-1H-isoindoL-4-yl]acetamide, or another product containing apremilast for the treatment of various inflammatory diseases. In some embodiments, the drug delivery device may contain or be used with Parsabiv™ (etelcalcetide HCl, KAI-4169) or another product containing etelcalcetide HCl for the treatment of secondary hyperparathyroidism (sHPT) such as in patients with chronic kidney disease (KD) on hemodialysis. In some embodiments, the drug delivery device may contain or be used with ABP 798 (rituximab), a biosimilar candidate to Rituxan®/MabThera™, or another product containing an anti-CD20 monoclonal antibody. In some embodiments, the drug delivery device may contain or be used with a VEGF antagonist such as a non-antibody VEGF antagonist and/or a VEGF-Trap such as aflibercept (Ig domain 2 from VEGFR1 and Ig domain 3 from VEGFR2, fused to Fc domain of IgG1). In some embodiments, the drug delivery device may contain or be used with ABP 959 (eculizumab), a biosimilar candidate to Soliris®, or another product containing a monoclonal antibody that specifically binds to the complement protein C5. In some embodiments, the drug delivery device may contain or be used with Rozibafusp alfa (formerly AMG 570) is a novel bispecific antibody-peptide conjugate that simultaneously blocks ICOSL and BAFF activity. In some embodiments, the drug delivery device may contain or be used with Omecamtiv mecarbil, a small molecule selective cardiac myosin activator, or myotrope, which directly targets the contractile mechanisms of the heart, or another product containing a small molecule selective cardiac myosin activator. In some embodiments, the drug delivery device may contain or be used with Sotorasib (formerly known as AMG 510), a KRAS^{G12C} small molecule inhibitor, or another product containing a KRAS^{G12C} small molecule inhibitor. In some embodiments, the drug delivery device may contain or be used with Tezepelumab, a human monoclonal antibody that inhibits the action of thymic stromal lymphopoietin (TSLP), or another product containing a human monoclonal antibody that inhibits the action of TSLP. In some embodiments, the drug delivery device may contain or be used with AMG 714, a human monoclonal antibody that binds to Interleukin-15 (IL-15) or another product containing a human monoclonal antibody that binds to Interleukin-15 (IL-15). In some embodiments, the drug delivery device may contain or be used with AMG 890, a small interfering RNA (siRNA) that lowers lipoprotein(a), also known as Lp(a), or another product containing a small interfering RNA (siRNA) that lowers lipoprotein(a). In some embodiments, the drug delivery device may contain or be used with ABP 654 (human IgG1 kappa antibody), a biosimilar candidate to Stelara®, or another product that contains human IgG1 kappa antibody and/or binds to the p40 subunit of human cytokines interleukin (IL)-12 and IL-23. In some embodiments, the drug delivery device may contain or be used with Amjevita™ or Amgevita™ (formerly ABP 501) (mab anti-TNF human IgG1), a biosimilar candidate to Humira®, or another product that contains human mab anti-TNF human IgG1. In some embodiments, the drug delivery device may contain or be used with AMG 160, or another product that contains a half-life extended (HLE) anti-prostate-specific membrane antigen (PSMA)×anti-CD3 BiTE® (bispecific T cell engager) construct. In some embodiments, the drug delivery device may contain or be used with AMG 119, or another product containing a delta-like ligand 3 (DLL3) CART (chimeric antigen receptor T cell) cellular therapy. In some embodiments, the drug delivery device may contain or be used with AMG 119, or another product containing a delta-like ligand 3 (DLL3) CART (chimeric antigen receptor T cell) cellular therapy. In some embodiments, the drug delivery device may contain or be used with AMG 133, or another product containing a gastric inhibitory polypeptide receptor (GIPR) antagonist and GLP-1R agonist. In some embodiments, the drug delivery device may contain or be used with AMG 171 or another product containing a Growth Differential Factor 15 (GDF15) analog. In some embodiments, the drug delivery device may contain or be used with AMG 176 or another product containing a small molecule inhibitor of myeloid cell leukemia 1 (MCL-1). In some embodiments, the drug delivery device may contain or be used with AMG 199 or another product containing a half-life extended (HLE) bispecific T cell engager construct (BITE®). In some embodiments, the drug delivery device may contain or be used with AMG 256 or another product containing an anti-PD-1×IL21 mutein and/or an IL-21 receptor agonist designed to selectively turn on the Interleukin 21 (IL-21) pathway in programmed cell death-1 (PD-1) positive cells. In some embodiments, the drug delivery device may contain or be used with AMG 330 or another product containing an anti-CD33×anti-CD3 BiTE® (bispecific T cell engager) construct. In some embodiments, the drug delivery device may contain or be used with AMG 404 or another product containing a human anti-programmed cell death-1(PD-1) monoclonal antibody being investigated as a treatment for patients with solid tumors. In some embodiments, the drug delivery device may contain or be used with AMG 427 or another product containing a half-life extended (HLE) anti-fms-like tyrosine kinase 3 (FLT3)×anti-CD3 BiTE® (bispecific T cell engager) construct. In some embodiments, the drug delivery device may contain or be used with AMG 430 or another product containing an anti-Jagged-1 monoclonal antibody. In some embodiments, the drug delivery device may contain or be used with AMG 506 or another product containing a multi-specific FAP×4-1BB-targeting DARPin® biologic under investigation as a treatment for solid tumors. In some embodiments, the drug delivery device may contain or be used with AMG 509 or another product containing a bivalent T-cell engager and is designed using XmAb® 2+1 technology. In some embodiments, the drug delivery device may contain or be used with AMG 562 or another product containing a half-life extended (HLE) CD19×CD3 BiTE® (bispecific T cell engager) construct. In some embodiments, the drug delivery device may contain or be used with Efavaleukin alfa (formerly AMG 592) or another product containing an IL-2 mutein Fc fusion protein. In some embodiments, the drug delivery device may contain or be used with AMG 596 or another product containing a CD3×epidermal growth factor receptor vIII (EGFRvIII) BiTE® (bispecific T cell engager) molecule. In some embodiments, the drug delivery device may contain or be used with AMG 673 or another product containing a half-life extended (HLE) anti-CD33×anti-CD3 BiTE® (bispecific T cell engager) construct. In some embodiments, the drug delivery device may contain or be used with AMG 701 or another product containing a half-life extended (HLE) anti-B-cell maturation antigen (BCMA)×anti-CD3 BiTE® (bispecific T cell engager) construct. In some embodiments, the drug delivery device may contain or be used with AMG 757 or another product containing a half-life extended (HLE) anti-delta-like ligand 3 (DLL3)×anti-CD3 BiTE® (bispecific T cell engager) construct. In some embodiments, the drug delivery device may contain or be used with AMG 910 or another product containing a half-life extended (HLE) epithelial cell tight junction protein claudin 18.2×CD3 BiTE® (bispecific T cell engager) construct.

Although the drug delivery devices, assemblies, components, subsystems and methods have been described in terms of exemplary embodiments, they are not limited thereto. The detailed description is to be construed as exemplary only and does not describe every possible embodiment of the present disclosure. Numerous alternative embodiments could be implemented, using either current technology or technology developed after the filing date of this patent that would still fall within the scope of the claims defining the invention(s) disclosed herein.

Those skilled in the art will recognize that a wide variety of modifications, alterations, and combinations can be made with respect to the above described embodiments without departing from the spirit and scope of the invention(s) disclosed herein, and that such modifications, alterations, and combinations are to be viewed as being within the ambit of the inventive concept(s).

1. A drug delivery device for delivering a medicament, comprising:
a pump first housing at least partially supporting and/or surrounding a fluid displacement assembly;
a pump second housing at least partially supporting and/or surrounding a drive component for driving the fluid displacement assembly;
an inlet fluid path configured to deliver medicament to the fluid displacement assembly;
an outlet fluid path configured to receive medicament from the fluid displacement assembly;
wherein the pump first housing and the pump second housing are removably coupled with each other.

2. The drug delivery device as in claim 1, wherein the pump first housing and the pump second housing are removably coupled with each other along a first axis by a first component and along a second axis by a second component, wherein the first axis and the second axis are generally perpendicular to each other.

3. The drug delivery device as in claim 2, wherein the first axis is parallel with an axis of the drive component and the second axis is generally perpendicular with the axis of the drive component.

4. The drug delivery device as in claim 2, wherein the first component is at least one magnet and the second component is at least one slot-tab arrangement.

5. The drug delivery device as in claim 4, wherein the second component includes a pair of tabs and a pair of slots that are configured to be slidingly engaged with each other.

6. The drug delivery device as in claim 1, further comprising at least one isolation mount coupled with the first housing and/or the fluid displacement assembly.

7. The drug delivery device as in claim 1, wherein, when coupled, at least a portion of the drive component is slidably received within at least a component of the fluid displacement assembly.

8. A drug delivery system for delivering a medicament, comprising:
a medicament container containing a medicament;
an inlet fluid path configured to receive the medicament from the medicament container;
an outlet fluid path configured to deliver the medicament to a patient; and
a drug delivery device positioned adjacent to the inlet fluid path and the outlet fluid path, the drug delivery device having:
a pump first housing at least partially supporting and/or surrounding a fluid displacement assembly;
a pump second housing at least partially supporting and/or surrounding a drive component for driving the fluid displacement assembly;
an inlet fluid path configured to deliver medicament to the fluid displacement assembly;
an outlet fluid path configured to receive medicament from the fluid displacement assembly;
wherein the pump first housing and the pump second housing are removably coupled with each other.

9. A drug delivery device for delivering a medicament, comprising:
a pump first housing at least partially supporting and/or surrounding a fluid displacement assembly;
a pump second housing at least partially supporting and/or surrounding a drive component for driving the fluid displacement assembly;
an inlet fluid path configured to deliver medicament to the fluid displacement assembly;
an outlet fluid path configured to receive medicament from the fluid displacement assembly;
wherein the pump first housing and the pump second housing are removably coupled with each other; and
wherein at least one of the pump first housing and the pump second housing includes a dampening component adjacent to an interface between the pump first housing and the pump second housing.

10. A drug delivery device as in claim 9, wherein the dampening component is an overmolded component.

11. A drug delivery device as in claim 9, wherein the dampening component has a different hardness than the pump first housing and/or the pump second housing.

12. A drug delivery device as in claim 9, further comprising at least one sensor positioned along the inlet fluid path and configured to measure an operational parameter; and
a controller workingly coupled with the at least one sensor and the drive component, wherein the controller is configured to adjust at least one parameter of the drive component based on input information received from the at least one sensor.

13. A drug delivery device as in claim 12, wherein the controller includes an encoder-fed, closed loop system

14. The drug delivery system as claim 13, further comprising an encoder board for determining measured drive speed and a motor model for determining a calculated drive speed;
wherein the controller receives input relating to the measured drive speed and the calculated drive speed; and
wherein the controller is configured to adjust the at least one parameter of the drive component based on the measured drive speed and the calculated drive speed.

15-54. (canceled)

55. The drug delivery system as in claim 8, wherein the pump first housing and the pump second housing are removably coupled with each other along a first axis by a first component and along a second axis by a second component, wherein the first axis and the second axis are generally perpendicular to each other.

56. The drug delivery system as in claim 55, wherein the first axis is parallel with an axis of the drive component and the second axis is generally perpendicular with the axis of the drive component.

57. The drug delivery system as in claim 55, wherein the first component is at least one magnet and the second component is at least one slot-tab arrangement.

58. The drug delivery system as in claim 57, wherein the second component includes a pair of tabs and a pair of slots that are configured to be slidingly engaged with each other.

59. The drug delivery system as in claim 8, further comprising at least one isolation mount coupled with the first housing and/or the fluid displacement assembly.

60. The drug delivery system as in claim 8, wherein, when coupled, at least a portion of the drive component is slidably received within at least a component of the fluid displacement assembly.
